(19) United States
(12) Patent Application Publication
(10) Pub. No.: US 2002/0087275 A1
(43) Pub. Date: Jul. 4, 2002

(54) Visualization and manipulation of biomolecular relationships using graph operators

(76) Inventors: Junhyong Kim, Hamden, CT (US); Shan Jiang, Trumbull, CT (US)

Correspondence Address: NEEDLE & ROSENBERG, P.C., The Candler Building, Suite 1200, 127 Peachtree Street, N.E., Atlanta, GA, 30303-1811, US

(21) Appl. No.: 09/918,938

(22) Filed: Jul. 31, 2001

Related U.S. Application Data
(60) Provisional application No. 60/221,707, filed on Jul. 31, 2000.

(51) Int. Cl. G01N 33/48; G06F 19/00

(52) U.S. Cl. 702/19; 702/27

(57) ABSTRACT

A system for analyzing and graphically visualizing biomolecular data, such as genomic data, is provided.

### CROSS-REFERENCE TO RELATED APPLICATIONS

This application claims benefit of U.S. Provisional Application No. 60/221,707, filed Jul. 31, 2000. Application Ser. No. 60/221,707, filed Jul. 31, 2000, is hereby incorporated herein by reference.

### FIELD OF THE INVENTION

The disclosed invention is generally in the field of analysis of biological relationships, and more specifically in the field of computational algorithms for representing and analyzing large and heterogeneous molecular biological data.

### BACKGROUND OF THE INVENTION

Genomics technology has become one of the main driving forces behind biomedical research. Information from genomics technology is increasing at an exponential pace. Simultaneously, the development of new technologies such as DNA microarrays, those of functional genomics, and automatic text retrieval, is greatly enriching the kinds of information available. The integration of gene expression data, sequence data, and genome annotation would greatly facilitate the utilization of genomics information by academic and commercial biotechnology enterprises. Accordingly, the synthesis and integration of these disparate sources of genomics data into a biologically meaningful information is an immediate and fundamental need.

Some sources of genomics information such as metabolic pathways traditionally are represented in graph form, where nodes or vertices represent genes, and edges or arrows represent some biological action between the genes. For example, the Enzyme Classification system is a hierarchical graph of enzymes related to each other by biochemical action. Other types of information, such as gene function classification, have implied graph relationships also.

However, new genomics technologies such as DNA microarrays are generating complex data with no canonical methods of analysis. Complexity in data derived from this technology results from both the extreme scale of the data (thousands of dimensions) and the uncertainty of the biological implications of measurements such as global gene expression levels. Thus a multi-pronged approach to data analysis using various statistical techniques and databases is required in order to achieve a synthesis of information.

The analysis of microarray gene expression data requires the clustering of genes into groups of comparable expression profiles across experiments, or the clustering of experiments into groups of similar expression patterns across genes. Hierarchical clustering (Eisen et al., Cluster analysis and display of genome-wide expression patterns. Proc Natl Acad Sci USA 95: 14863-8 (1998)) and self-organizing maps (SOM) (Tamayo et al. (1999) Interpreting patterns of gene expression with self-organizing maps: methods and application to hematopoietic differentiation. Proc. Natl. Acad. Sci. USA, 96:2907-2912) currently are the algorithms used most commonly for expression data clustering, and are implemented in a number of shareware and commercial software products. The most salient disadvantage of hierarchical clustering is that each individual gene occupies a unique position in the hierarchical tree, and cannot be assigned to more than one group. The SOM algorithm requires an arbitrary predetermination of the number of clusters to be formed, and thus may yield clusters of suboptimal quality.

In order to overcome the disadvantages of conventional algorithms, several new algorithms based on graph theoretic tools have been proposed recently. Ben-Dor et al. (1999) Clustering gene expression patterns. J. Comput. Biol., 6(3/4): 281-297, describe a clustering algorithm using graph theoretic framework in combination with a probabilistic model. They devised an algorithm to generate a clique graph from the similarity matrix derived from gene expression data. Input data are represented in a disconnected undirected graph in which each gene corresponds to a vertex. A clique graph, defined as a disjoint union of complete graphs, represents a possible clustering of vertices. This algorithm produces nonhierarchical clusters, the number of which is determined by the probabilistic algorithm.

Another algorithm for expression data clustering was proposed by Sharan and Shamir, (2000) CLICK: A clustering algorithm with applications to gene expression analysis. ISMB 2000, 307-316, using the graph representation and a statistical model. As in the algorithm elaborated by Ben-Dor et al (1999), data elements are represented by vertices of a graph. The computation starts from a complete graph, and generates multiple subgraphs/clusters by recursively cutting each edge whose weight falls into the statistically non-connected category.

The third algorithm based on graph theory for analyzing expression data, biclustering, was developed by Cheng and Church, (2000) Biclustering of expression data. ISMB 2000, 93-103. In this algorithm, genes and experiments are represented as vertices of a bipartite graph, and are clustered simultaneously. The mean square residue score of the data matrix for each cluster is used as a measurement of the coherence of gene expression across experiments. The algorithm is designed to find a maximum complete bipartite sub-graph with the lowest mean square residue score. The result of this computation is a set of gene-experiment clusters in which the expression of the genes is coherent across the experiments. Thus, the biclustering algorithm creates multiple overlapping clusters that better represent genes that participate in multiple pathways.

Although the algorithms summarized above provide solutions for primary data analysis, they do not address the need for comparison, integration, and data mining of multiple disparate genomic data sets. To address this need, some data integration efforts such as KEGG (Kyoto Encyclopedia of Genes and Genomes) (Kanehisa and Goto, (2000) KEGG: Kyoto encyclopedia of genes and genomes. Nucleic Acids Research, 28:27-30; Ogata et al. (1998) Analysis of binary relations and hierarchies of enzymes in the metabolic pathways. Biosystems, 47: 119-128; Kanehisa et al. (2000) Functional enzyme clusters. Nucleic Acids Research, 28:27-30) and DIP (The Database of Interacting Proteins) (Marcotte et al. (1999) A combined algorithm for genome wide prediction of protein function. Nature, 402: 83-86; Xenarios et al. (2000) DIP: the database of interacting proteins. Nucleic Acids Research, 28:289-91) databases have endeavored to integrate into pathways gene relationships previously expressed in binary form. However, the computations in these systems were carried out at the database level by querying a database for all potential consecutive binary gene pairs, and subsequently, integrating them into pathways. Computations carried out within the database framework are limited to some relatively simple analyses such as the generation of pathways, and coloring genes in the pathway. More complex analyses such as comparing disparate data sets, exploring gene network structures, and inferring pathways and gene functions, are either beyond the capacity of these systems or computationally too expensive to perform.

### BRIEF SUMMARY OF THE INVENTION

Disclosed is a method for universal representation and integration of heterogeneous molecular biological relationships using graph theoretic tools. The disclosed invention relates to an electronic system, computer-implemented method, and program product in which graphs are stored, manipulated and/or graphically output on a display or other output device. Biological molecules are represented as vertices in the disclosed graphs. Edges that connect vertices in the graph represent the presence of relationships between the molecules. The edge weight of the edges contains quantitative or qualitative descriptions of the relationship. Thus, molecular biological data of different sources and natures can be represented under a single unified structure that provides the foundation for integration of disparate molecular biological data. FIG. 1 exemplifies the basic components of the disclosed molecular relational graphs. Moreover, a complete suite of abstract operations and associated rules are defined for the graph such that any specific computation of the disclosed method can be achieved by compounding operations according to the rules. Thus operations and rules defined for the graph confer powerful tools for assimilating disparate molecular biological data.

The disclosed method relates to the application of graph theoretical data representation coupled with graph operators to biomolecule data analysis. This analysis framework is referred to herein as the “molecular relational graphing” (MRG) data model or as the “gene-graph operator” (GGO) data model. Using the MRG model, analysis techniques for synthesis of disparate sources of knowledge such as those of microarray gene expression, protein-protein interaction, and gene function can be developed. In some embodiments, the disclosed method relates to the application of graph theoretical data representation coupled with graph operators to genomic data analysis.

It is an object of the present invention to provide a system for analyzing and graphically visualizing genomic data.

It is another object of the present invention to provide a comprehensive model to organize and store gene relationship information as graphs.

It is another object of the present invention to provide algorithms to analyze and compare molecular relational graphs.

It is another object of the present invention to provide a software program to implement a molecular relational graphing data model.

It is another object of the present invention to provide a software program to visualize the molecular relational graph data.

It is another object of the present invention to provide a large database for the storage and organization of molecular relational graphing data.

It is another object of the present invention to provide an integrative user operation environment based on a graphical flowchart metaphor.

### BRIEF DESCRIPTION OF THE DRAWINGS

FIG. 1 is a diagram showing an example of the basic structure of the disclosed graphs.

FIG. 2 shows a gene-graph (or molecular relational graph) of protein-protein interactions in yeast. Data were generated by yeast two-hybrid assay (Uetz et al., 2000). Each gene is represented as an oval and the interactions between two genes is represented by the line connecting the two ovals. This graph encompassed 1,004 genes and 957 interactions. Approximately 500 genes form the largest interconnected structure. The rest form a number of smaller structures.

FIG. 3 shows a gene-graph (or molecular relational graph) of gene ontology functional relationships for a selected set of yeast genes. Thirty-one genes are included in this graph. Their participation in multiple functional processes makes the intersecting pathways form a dense network.

FIG. 4 shows a gene-graph (or molecular relational graph) of expression analysis data. Data were from a correlation analysis of microarray hybridization experiments reported by Spellman et al. (1998). Edges in the graph represent the correlation between two genes in gene expression profile. This graph is derived by edge-thresholding at 0.4. This graph is generated from correlation analysis of yeast gene expression profile during cell cycle.

FIGS. 5A, 5B, 5C, 5D, and 5E show a gene-graph analysis (or molecular relational graphing analysis) of expression data from microarrays hybridizations assay. FIG. 5A shows the gene-relationship structure derived by applying the AND operator between the Gene Ontology (GO) annotation graph and the gene expression graph, wherein both graphs have the same graph structure. Two structures are labeled as *1 and *2, respectively. FIG. 5B shows the expression gene-graph threshold at 0.1. Both structure *1 and *2 are present, some relationships are missing in structure *1 due to the high-stringency thresholding. One novel structure (∇) cannot be derived from naive GO annotation grouping. However, it is supported by the sophisticated grouping as shown in FIG. 5E. FIG. 5C shows an expression gene-graph thresholded at 0.2. Both structure *1 and *2 are completely preserved, and the novel structure ∇ is expanded by the addition of one gene and two new relationships. FIG. 5D shows an expression gene-graph thresholded at 0.3. Structure *1 is completely preserved while *2 is expanded into a larger one with additional genes and relationships. Structure ∇ is expanded also and a fourth structure appears in the graph. FIG. 5e shows the relative positions of two GO id numbers GO:0007330 and GO:0007328 in GO annotation tree. This GO genealogy clearly indicates the legitimacy of the relationship that forms the structure ∇.

FIG. 6 is a diagram of an overview of an example of the design of a data mining system using the disclosed method.

FIG. 7 is a diagram of an example of the design of a data mining service client.

FIG. 8 is a diagram of an example of the design of a data mining service broker.

FIG. 9 is a diagram of an example of the design of a graph computation manager.

FIG. 10 is a diagram of an example of the design of a graph computation engine.

FIG. 11 is a diagram of an example of the design of a graph visualization engine.

FIG. 12 is a diagram of an example of the design of a graph computational library.

FIG. 13 is a diagram of an example of the design of a data interface.

FIG. 14 is a diagram of an example of a general purpose computer implementing an example of the disclosed method and composition.

FIG. 15 shows a Unified Modeling Language diagram of GGO (or MRG) objects.

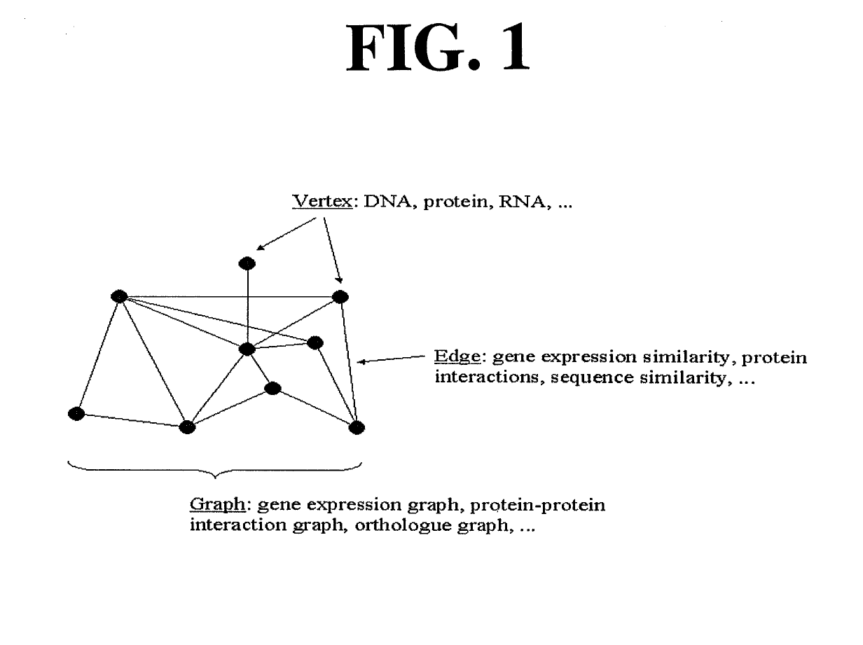

### DETAILED DESCRIPTION OF THE INVENTION

Disclosed is a method for universal representation and integration of heterogeneous molecular biological relationships using graph theoretic tools. In the method, biological molecules can be represented as vertices in the graph. Edges that connect vertices in the graph can represent relationships between molecules. Edge weight can contain quantitative or qualitative descriptions of the relationship. In this way, molecular biological data of different sources and natures can be represented under a single unified structure that provides the foundation for integration of disparate molecular biological data. Moreover, a complete suite of abstract operations and associated rules can be defined for, and applied to, the graph such that any specific computation of the disclosed method can be achieved by compounding operations according to defined and devised rules. Thus, operations and rules defined for the graph confer powerful tools for assimilating disparate molecular biological data.

The disclosed method is referred to herein as molecular relational graphing (MRG) and involves generation and manipulation of graphs, referred to herein as molecular relational graphs. Alternatively, the method is referred to as gene-graph operator (GGO) and the graphs are referred to as gene-graphs.

The disclosed method can be implemented as computer software. For example, a molecular relational graphing software program can be written using any suitable programming language, such as the Java™ programming language. A software program implementing the disclosed method can have two principal features: (1) implementation of molecular relational graphing objects and the ability to store in a local and/or remote database, and (2) implementation of operators. Such operators manipulate the molecular relational graphs as objects, much as mathematical operators manipulate numbers. Like mathematical operators, molecular relational graphing operators allow direct manipulation of graphs using graph operations such as addition and subtraction.

Molecular relational graphing is preferably implemented on a programmed general purpose computer system. However, the molecular relational graphing can also be implemented on a special purpose computer, a programmed microprocessor or microcontroller and peripheral integrated circuit elements, an ASIC or other integrated circuit, a hardwired electronic or logic circuit such as a discrete element circuit, a programmable logic device such as a PLD, PLA, FPGA or PAL, or the like.

The disclosed molecular relational graphing method provides a comprehensive framework to accommodate disparate data sets; the underlying graph theoretic tools confer powerful approaches, for example, to analyze network structures, and to infer pathways and functions. The method complements existing integrative efforts. Most importantly, the integrative and analytical capacity of the disclosed molecular relational graphing is far greater than that of any existing algorithm.

The disclosed method provides a new technique for genomics data analysis, including that generated by microarrays. In the disclosed method, heterogeneous genomics information can be unified into a common graph-theoretic structure. Subsequently, formal graph operators can be defined, allowing the manipulation of different information through a syntax of graph structures. The disclosed method allows querying of complex information with a dynamic rearrangement and synthesis of heterogeneous data.

The disclosed method offers a universal representation of heterogeneous molecular biological data. Biological data of different sources can be captured in a single unified structure based on intermolecular relationships. Modification and integration of heterogeneous data are achieved by applying single or compounded operations on multiple data sets. Thus, unlike previous techniques, the disclosed method is not restricted to any particular problem domain and is not limited to a few fixed kinds of data integration. As used herein, heterogeneous biological data, heterogeneous molecular biological data, or heterogeneous biomolecular data refers to data from different types of biological systems (thus embodying different types of relationships between biological molecules), different types of measurements (thus embodying different types of relationships between biological molecules), different types of biological molecules (preferably different types of biological molecules that have relationship with each other), or any other combination of disparate biological data. As an example, one form of heterogeneous molecular biological data would be expression relationships between genes and proteins (two different types of biological molecules). Another form of heterogeneous molecular biological data would be the combination of a variety of expression and physiological measurements (that is, multiple different relationship nd biological molecules) for a particular type of cell or tissue.

Different types of biological systems include, for example, protein-protein interactions; protein-nucleic acid interactions; gene expression regulation; protein expression regulation; cellular signal transduction pathways; physiological states; disease states; and metabolic pathways. Different types of measurements include, for example, the presence of association in time, or space, or logical meaning; physical or logical states such as activation and inhibition; real value measurement of spatial distance such as physical distances between genes, open reading frames, single nucleotide polymorphisms, expressed sequence tags, sequence tag sites, or a combination thereof; sequence similarity between genes or proteins; structural similarity between proteins; radiation hybrid mapping distances between genes, open reading frames, single nucleotide polymorphisms, expressed sequence tags, sequence tag sites, or a combination thereof; genetic distances between genes, open reading frames, single nucleotide polymorphisms, expressed sequence tags, sequence tag sites, or a combination thereof; real value measurement of time or kinetic information such as chemical conversion rate; Euclidean and other distance metrics in feature space to measure logical relationship; correlation coefficient as a statistical metric to measure logical relationship; values of fuzzy set membership function as a metric to measure logical relationship; and conditional probability as a measurement of causal relationship.

Different types of biological molecules include, for example, genes, open reading frames, expressed sequence tags, single nucleotide polymorphisms, sequence tag sites, nucleic acids, DNA, RNA, mRNA, cDNA, proteins, peptides, enzymes, metabolites, carbohydrates, exons, introns, cleavage fragments, restriction fragments, amino acid modifications, protein domains, DNA or RNA secondary or tertiary structures, nucleic acid motifs, protein motifs, and metal ions.

In the context of the disclosed molecular relational graphs, use of heterogeneous molecular biological data is manifested by having at least two of the vertices represent different types of biological molecules; having at least two edges represent different types of relationships between the biological molecules represented by the vertices connected by the edges; having at least one edge represent a plurality of different types of relationships between the biological molecules represented by the vertices connected by the edge; and/or having at least one vertex represent a plurality of different types of biological molecules.

A graph is a mathematical abstraction of relationships among different entities in the real world. The graph represents an entity (such as a gene, protein, or other biomolecule) as a vertex, and encapsulates the relationship between two entities as an edge that connects the two vertices. The interconnections among a set of vertices, designated by a set of edges, form a graph. Many algorithms have been developed that allow efficient manipulation of the graph, retrieval of information stored in the graph, and computation using graphs as objects. Graph theory and techniques can be applied, in the disclosed method, to model and manipulate biomolecules and biological relationships organized as a graph.

The disclosed method relates, in part, to the application of the gene-graph operator method to the analysis of genomic relationships. Genomic relationships can be encapsulated by a graph model regardless of the context and the technology from which the information is derived. In GGO, each gene (or protein or biomolecule) is represented as a vertex in the graph, and the relationship between two genes (or proteins or biomolecules) is represented as the edge between vertices. The graph model can be used to represent various types of genomic relationships (or other biomolecular relationships) as defined by the contents of the vertex and the edge. For example, a graph can model a gene expression data set if the edge contains the measurement of correlation of the expression patterns of two genes. With such a gene-graph model, algorithms developed in graph theory enable sophisticated analysis of the gene-relationship data. Examples of complex analysis include the elucidation of mechanisms of gene regulation, the identification of gene action pathways, and the identification of critical genes that link multiple biochemical pathways.

In some embodiments, the disclosed method can use and manipulate large databases, including object-oriented databases, for the storage and organization of molecular relational graph data (or gene-graph data), and can implement molecular relational graphing models for proteome and genome mapping data. A molecular relational graphing database can comprise large data sets from a variety of sources, such as gene expression analysis, proteome analysis, genome mapping, and functional genome annotation. Data objects, n-nary operations, and graph functions can be implemented as, for example, individual software components, which then can be connected to implement a particular set of analysis operations. The software components can be graphically represented as iconized tools. Connections between components can be established by the user from a graphical interface.

The manipulations of graphs in the disclosed method may involve single graphs (by using unary operators) or multiple graphs (by using binary and n-nary operators), and may produce numerical results or new graphs (referred to herein as product graphs). These manipulations can be designed such that they can be combined into a sequence of steps to produce a particular synthetic meta-analysis. The manipulations can also be recursive, with, for example, a result of a manipulation being manipulated again (or multiple times) in the same way. The results of the meta-analysis can be interpreted in a biological context. In other words, instead of fixing the results of, for example, microarray analyses or various genomics information into a static and awkward data model, the information can be encapsulated into a common graph structure with associated syntactic rules that are defined for manipulating the common structure. This encapsulation produces an information model that is dynamic and particularly suited to synthesis of disparate information.

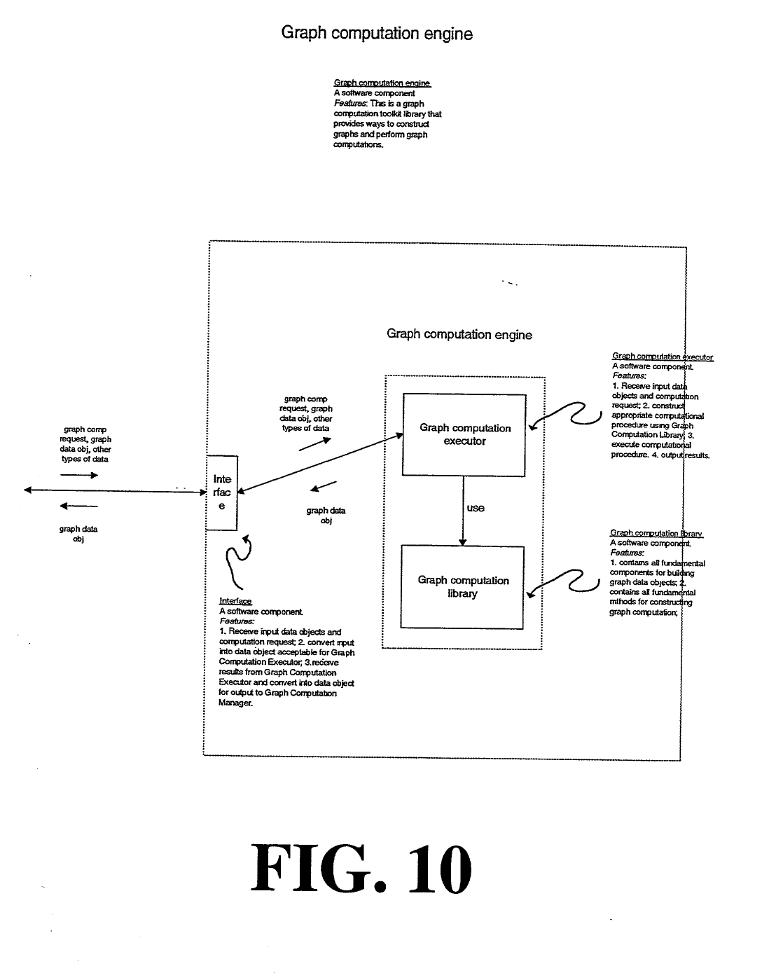

The disclosed method and composition can be understood further by reference to the following example system, which describes an example of the use of a gene graph operator (which is also referred to as a molecular relational graphing operator) at the heart of a data mining and interface system. The gene graph operator (FIG. 12) is a software embodiment of the disclosed method and provides representations for all types molecular relational graphs (gene-graphs). The gene graph operator is used by the graph computation executor in the graph computation engine (FIG. 10) to construct molecular relational graphs and perform operations on molecular relational graphs.

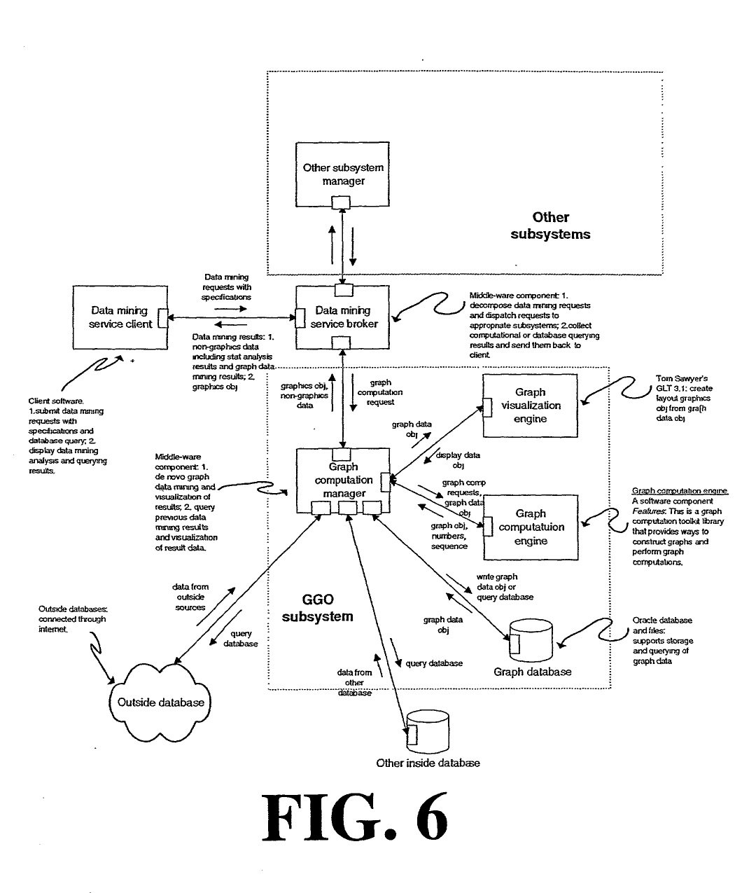

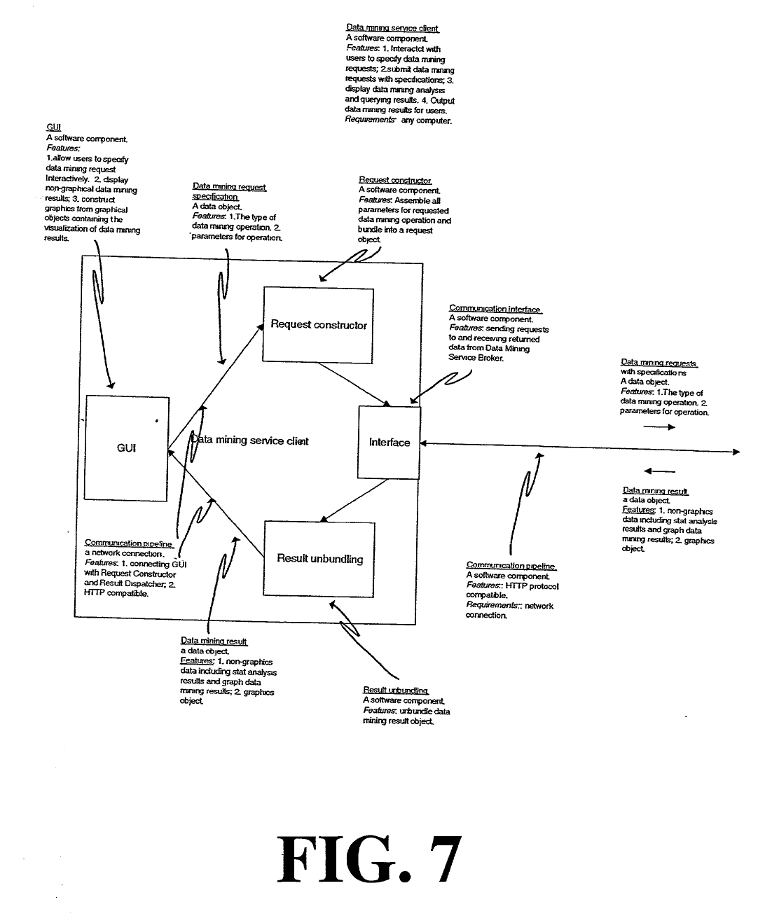

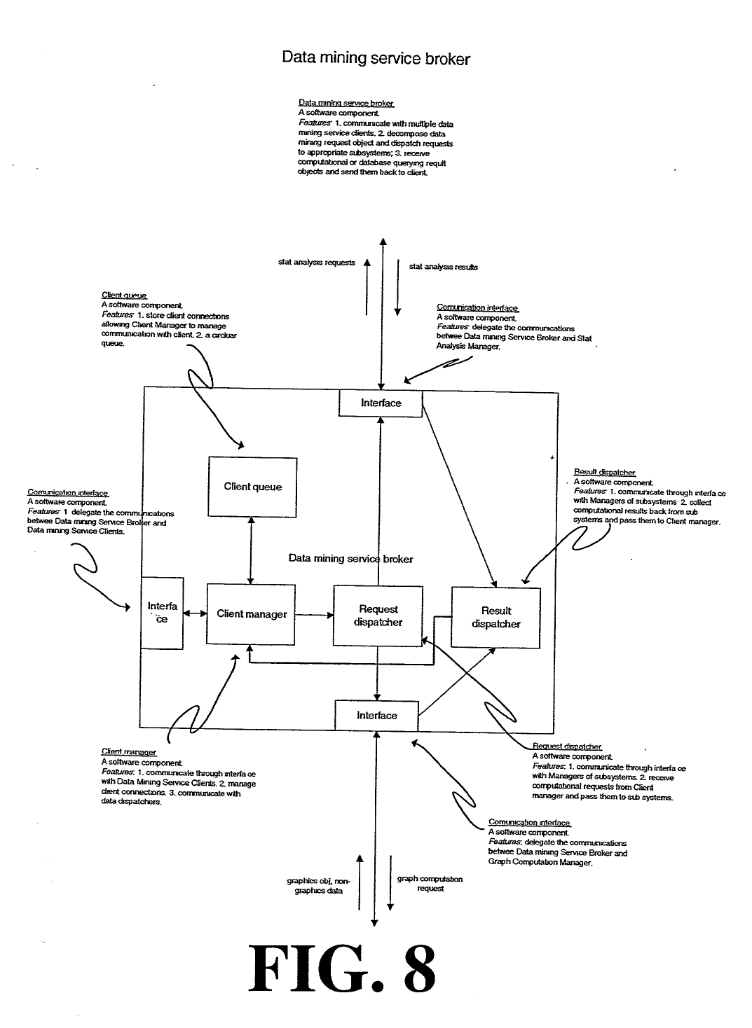

As illustrated in FIG. 6, the user can submit a data mining request by interfacing with the data mining service client (details in FIG. 7). The data mining service client includes the user interface and displays results of data mining and graph manipulation (FIG. 7). The data mining service client then makes a data mining request of the data mining service broker (details in FIG. 8). The data mining service broker decomposes data mining requests and dispatches requests for data to various subsystems. The data mining service broker also communicates the results of data mining, graph construction, and graph manipulation to the data mining service client.

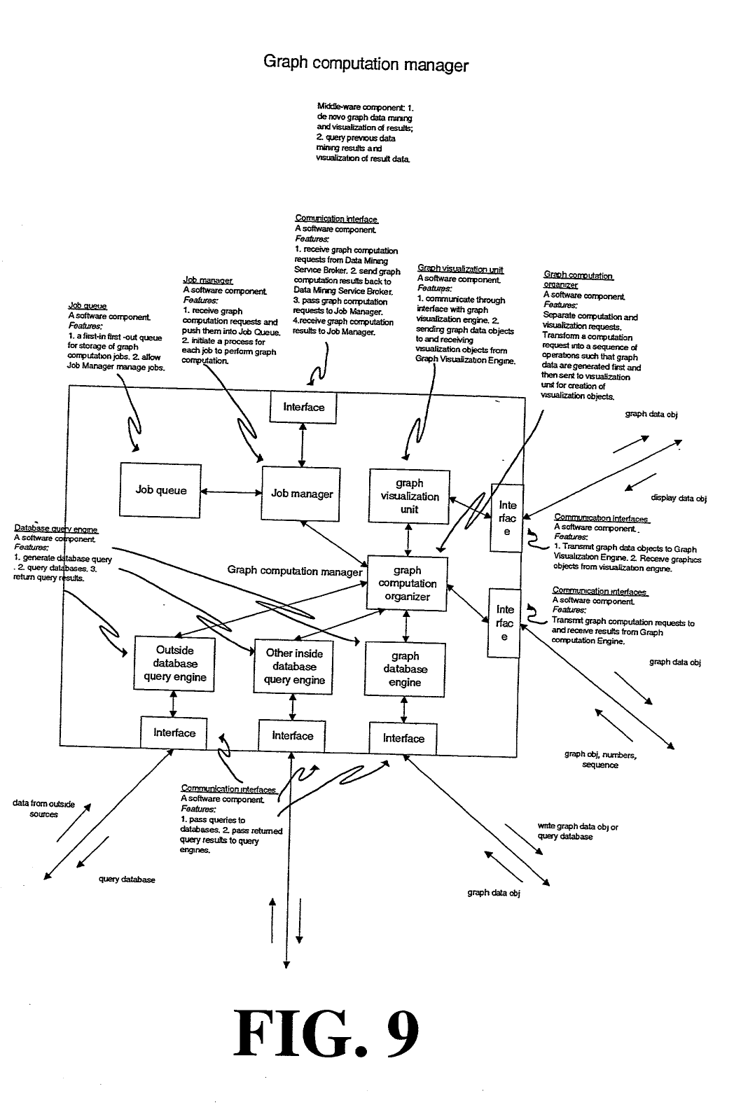

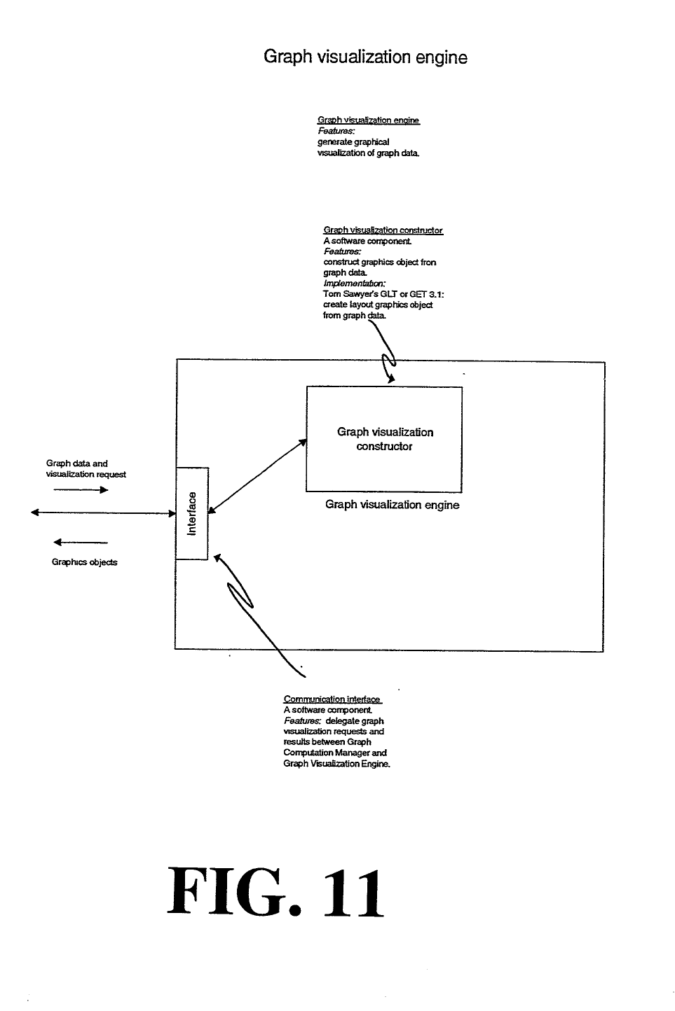

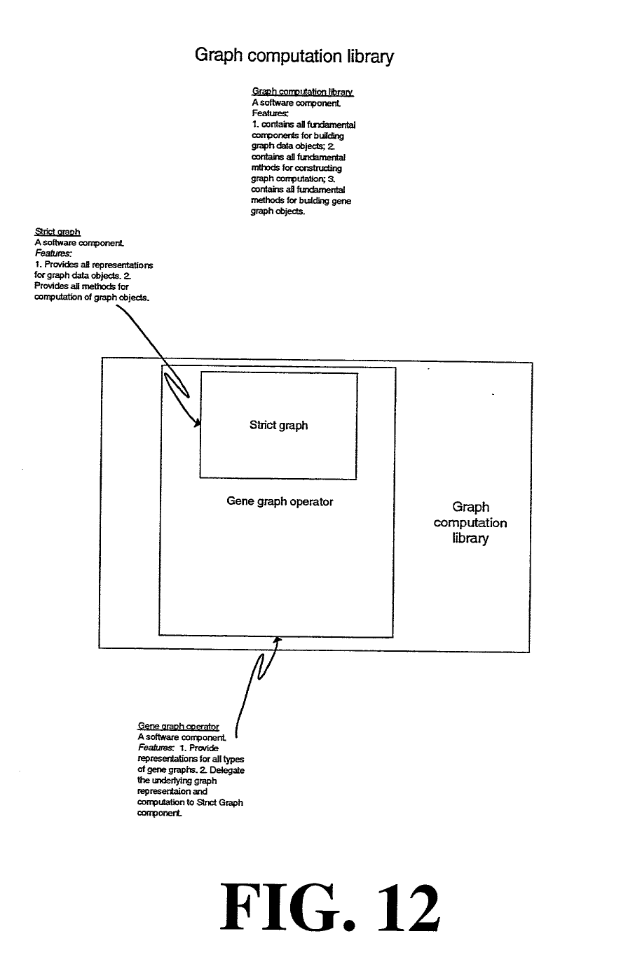

As illustrated in FIG. 6, the data mining service broker makes graph computation requests to the graph computation manager (FIG. 9). The data mining services broker also receives the results of data mining, graph construction, and graph manipulation from the graph computation manager (FIG. 6). The graph computation manager interfaces with databases to receive graph data (FIG. 6). The graph computation manager sends graph computation requests to the graph computation engine (FIG. 10). The graph computation engine builds graphs from the data received from the graph computation manager and performs operations on graphs. The results of the computations are communicated to the graph computation manager (FIG. 6). The graph computation manager also sends graph visualization requests to the graph visualization engine (FIG. 11). The graph visualization engine produces graphics objects from graph data and communicates the graphics objects to the graph computation manager (FIG. 6). The graph computation manager sends the graphics objects and non-graph data from data mining operations to the data mining service broker which in turn communicates the non-graph data and graphics objects to the data mining service client where the user can access and view the results (FIG. 6).

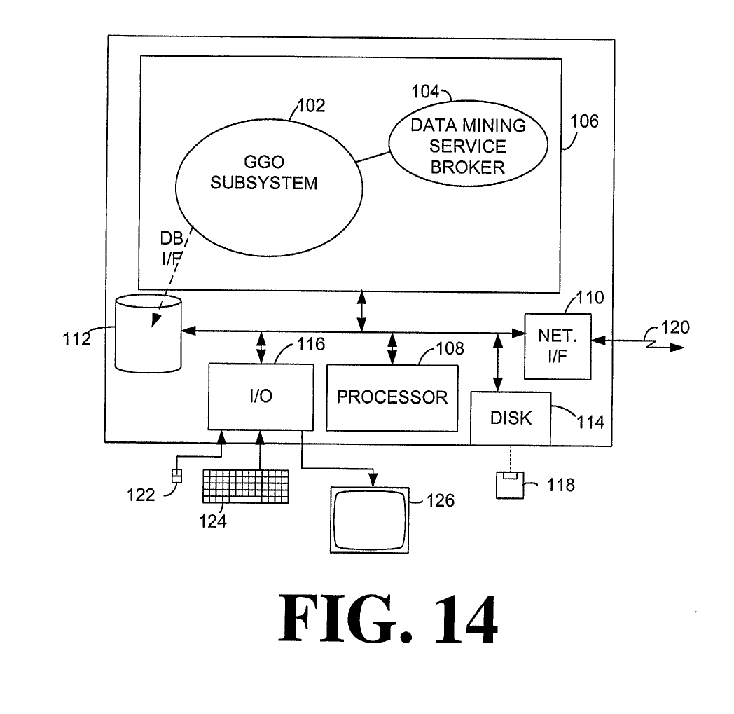

The disclosed method and composition can be understood further by reference to the following example system. As illustrated in FIG. 14, the user can load data and interact with the system through network interface 110, disk 118 and 114, keyboard 124, or a combination. The user graph data can be formatted as flat files of ASCII or binary type; files with fields separated by comma, tab, line break, carriage return, or paragraph or other character codes for import into spreadsheets. A preferred format is appropriate tables of a relational database. The graph data can be accessed by a graph manipulation component such as GGO subsystem 102 (see also FIG. 6). The GGO subsystem can obtain graph data by request from the data mining service broker 104 (see also FIG. 8). The system can display for the user visual representations of graph data on monitor 126 or other display device.

To adapt graph structures to the analysis of biomolecule relationship data, graph theoretical vocabulary can be defined in a biological context. Using this vocabulary, biomolecular relationship information, such as information derived from gene expression analysis or the Gene Ontology (GO) database, can be represented and integrated using the disclosed molecular relational graphing model.

Accordingly, for purposes of the disclosed method, by “graph” it is meant a collection of vertices (nodes) and edges denoted as G={V, E} where V is the set of vertices and E is the set of edges.

By “vertex” and “vertices” it is meant an encapsulation representing a biological molecule such as DNA, RNA, protein, or small compounds. Vertices can be labeled with the identities of the biological molecules. If two different graphs share identically-labeled vertices (or one or more allowed aliases), it is assumed, unless the context is to the contrary, that they are comparable. For example, a vertex in a gene expression graph might be labeled “CDC28” and a vertex in a protein-protein interaction graph might also be labeled “CDC28”. They are assumed to be comparable even though the actual molecules in the experiments might not be identical. Vertices can encapsulate all the properties of the biological molecules, and therefore, may be multi-labeled.

By “hyper-vertex” it is meant a set of vertices representing a set of biological molecules. Unless the context clearly indicates otherwise, the term “vertex” is used herein to refer to both vertices as defined above and hyper-vertices.

By “edge” is it meant a connection between two vertices. It usually represents a relationship between the biological molecules specified by the two vertices. An edge can be directed, representing the direction of action, and it can be weighted. An edge can be said to be defined by a pair (a, b) where a and b each represent a vertex.

By “edge weight” it is meant a number or a descriptor assigned to an edge, denoting a quantitative degree of relationship or qualitative type of relationship. For example, a real-valued edge weight can denote the correlation coefficient between expression patterns of two genes; an edge weight with the descriptor “+” can denote “activation” of one gene by another.

By “hyper-edge” it is meant an edge which connects two or more vertices as a set denoting a relationship that involves more than pair-wise interactions. A hyper-edge may also be weighted. A hyper-edge can be said to be defined by a pair (a, b) where at least one of a and b represents a set of vertices. For a regular hyper-edge, both a and b represent a set of vertices. Unless the context clearly indicates otherwise, the term “edge” is used herein to refer to both edges as defined above and hyper-edges.

By “directed edge” it is meant an edge defined as an ordered pair (a, b) where a and b are vertices.

By “undirected edge it is meant an edge defined as an unordered pair (a, b) where a and b are vertices.

By “directed hyper-edge” it is meant a hyper-edge defined as an ordered pair (a, b) where a and/or b are sets of vertices.

By “undirected hyper-edge it is meant a hyper-edge defined as an unordered pair (a, b) where a and/or b are sets of vertices.

In some embodiments, the disclosed software can perform the task of integrating data from, for example, microarray gene expression analysis, Gene Ontology annotation, and protein-protein interaction analysis into a molecular relational graphing data model. The disclosed software can also have functions for pathway analysis, critical gene identification, gene-action subsystem identification, and pathway comparison. Since the molecular relational graphing model is best illustrated using a graphical approach, also disclosed is visualization software for the demonstration of data resulting from computation using the disclosed molecular relational graphing data model. Such software can be written in any suitable programming language, for example, the Java programming language.

Graph objects, n-nary operators, and graph operators can be implemented as individual software components, which are then connected in series using connectors to implement the desired set of analysis operations. The software components and connectors can be graphically represented as intuitively recognizable glyphs. The user of the software can establish connections between components by using the graphical interface. Standard analysis techniques can be integrated into the disclosed analysis platform by incorporating standard commercial software packages. This will allow the system to use many analysis features from other packages, such as clustering analysis, for preliminary data processing. The resulting data can be transformed into the molecular relational graphing model for high-level analysis.

In some embodiments, molecular relational graphing models for proteome and genome mapping data will be used. In such embodiments, the molecular relational graphing database can contain large data sets from gene expression analysis, proteome analysis, genome mapping, and/or functional genome annotation.

A. Graph Elements

The disclosed method uses graphs to embody and manipulate relationships between biomolecules. Heterogeneous molecular biological relationships can be effectively encapsulated in different molecular relational graphs. In a molecular relational graph, biological molecules are represented by vertices and information of relationships between molecules is stored in edges connecting vertices.

1. Vertices

Different types of biological molecules can be represented as different types of vertices in molecular relational graphs. Biological molecules that can be represented by vertices in molecular relational graphs include but are not limited to:

genes, open reading frames, expressed sequence tags, single nucleotide polymorphisms, sequence tag sites, nucleic acids, DNA, RNA, mRNA, cDNA, proteins, peptides, enzymes, metabolites, carbohydrates, exons, introns, cleavage fragments, restriction fragments, amino acid modifications, protein domains, DNA or RNA secondary or tertiary structures, nucleic acid motifs, protein motifs, and metal ions.

As used herein, “biological molecule” and “biomolecule” refer to any molecule or portion of a molecule or multi-molecular assembly or composition, that has a biological origin, is related to a molecule or portion of a molecule or multi-molecular assembly or composition that has a biological origin. Biomolecules can be completely artificial molecules that are related to molecules of biological origin.

The content of a vertex can include a label and an information table. To construct a vertex, a name that uniquely labels a biological molecule can be used as the label for the vertex. Properties of the biological molecule can be stored in an information table as a part of the content possessed by the vertex such that each row of the table contains a property name and a property value.

Using information retrieved from the Sacchoromyces Genome Database (SGD) (Cherry et al., Sacchoromyces Genome Database), the following illustrations provide examples of constructing vertices representing yeast open reading frames (ORFs), protein molecules, and genes.

Illustration 1: Defining Vertices Representing Yeast Open Reading Frames (ORFs)

More than 5,000 genes were identified in yeast genome by either experimental or computational methods (Cherry et al. (1997)). Each gene consists of one or more exons in its genomic sequence that, when spliced together in order, forms the sequence of mRNA for this gene. Part of the mRNA molecule will be translated into proteins. The translated portion of the mRNA molecule sequence does not contain any translational stop codon. Thus, a continuous fragment of genomic sequence, which constitutes a part or whole of translated portion of an mRNA molecule, can be named an open reading frame (ORF).

To construct vertices representing yeast ORFs (Cherry et al. (1997)), a unique label for a vertex can be specified, for example, using the name of the ORF such as “YCL040W”. A vertex can also possess an information table in which properties of the represented yeast ORF can be stored. The information table can have two columns: <property_name>and <value>. The content of the table can comprise a set of (property_name, value) pairs that can include, for example: alias, chromosome_location, genomic_sequence_source, description, gene_product, function, cellular_component, process, and phenotype. Table 1 shows the content and structure of the information table for a vertex representing a yeast ORF, YCL040W.

TABLE 1
Information table for a vertex representing yeast ORF YCL040W.

| Property_name | Value |
|---|---|
| Alias | GLK1 |
| chromosome_location | chromosome_3 |
| genomic_sequence_source | SGD_YCL040W |
| Description | Glucose phosphorylation |
| gene_product | Glucokinase |
| Function | Glucokinase |
| Cellular_component | Cytosol |
| Process | Glycolysis |
| Phenotype | Null mutant is viable with no |
| | discernible difference from wild- |
| | type; hxk1, hxk2, glk1 triple null |
| | mutants are unable to grow on any |
| | sugar except galactose and fail to |
| | sporulate. |

Illustration 2: Defining Vertices Representing Yeast Proteins

To represent yeast protein molecules using vertices, one vertex can represent one protein molecule. In this representation, the label of a vertex can be assigned the name of the represented protein molecule. An information table can be constructed for each vertex. The table can comprise two columns: <property_name>and <value>. A list of (property_name, value) pairs can be stored in the table. In the information table possessed by different vertices, the same property_name may be associated with different values. The list of property_names can include, for example: alias, sequence_source, structure, EC_number, description, function, cellular_component, process, and phenotype. An information table for a vertex representing yeast protein grx1 is shown in Table 2. The label of the vertex is GRX1.

TABLE 2
Information table for a vertex representing yeast protein grx1.

| Property_name | Value |
|---|---|
| sequence_source1 | PID_G5328 |
| sequence_source2 | SwissProt_P25373 |
| sequence_source3 | PIR_S19363 |
| Structure | Sacch3D_YCL035C |
| Description | Glutaredoxin |
| Function | Glutaredoxin |
| cellular_component | Unknown |
| Process | oxidative stress response |
| Phenotype | Null mutant is viable but sensitive |
| | to oxidative stress. grx1 grx2 null |
| | mutants are viable but lack heat- |
| | stable oxidoreductase activity |

Illustration 3: Defining Vertices Representing Yeast Genes

A complete representation of yeast genes can consist of information for both the genomic sequence and the protein products of the gene. By merging together information contained in vertices representing the ORFs of a gene and the corresponding protein products, a vertex that represents the gene can be constructed. To create a vertex representing a yeast gene, given that a vertex (vertices) representing the ORF(s) of the gene and a vertex (vertices) representing the protein product(s) of the gene are created previously, a series of operations can be performed. For example:

Assign the name of the gene to the label for the vertex.

Create an information table for the vertex.

Add (property_name, value) pairs (ORF, ORF_name) to the table. ORF_name is the label for a merged-in vertex representing an ORF. There may be several (ORF, ORF_name) pairs if the gene encompasses more than one ORF.

Add the second type of (property_name, value) pairs, (protein, protein_name), to the table. Protein_name is the name of the merged-in vertex representing a protein molecule. There may be several (protein, protein_name) pairs if the gene is translated into protein molecules of more than one isoform.

Add additional (property_name, value) pairs to the table such that each pair consists of the label of a merged-in vertex and the information table possessed by the corresponding vertex.

As an example, a vertex representing a yeast gene, GRX1, is created from a vertex representing an ORF, YCL035C, and a vertex representing a protein molecule, grx1. Since the gene contains only a single ORF and a single protein product, there is only one ORF vertex and one protein vertex participating in the construction of the vertex representing the gene. The label of the vertex representing the gene is specified as GRX1. The information table for the vertex is shown in Table 3.

TABLE 3
Information table for a vertex representing yeast protein grx1.

| Property_name | Value |
|---|---|
| ORF1 | YCL035C |
| Protein | grx1 |

YCL035C

| | |
|---|---|
| chromosome_location | chromosome_3 |
| Sequence coordination | 61173 to 60841 |
| genomic_sequence_source | SGD_YCL035C |
| Description | Glutaredoxin |
| gene_product | Glutaredoxin |
| Function | Glutaredoxin |
| Process | oxidative stress response |
| Phenotype | Null mutant is viable but sensitive to |
| | oxidative stress. grx1 grx2 null |
| | mutants are viable but lack heat- |
| | stable oxidoreductase activity. |

GRX1

| | |
|---|---|
| sequence_source1 | PID_G5328 |
| sequence_source2 | SwissProt_P25373 |
| sequence_source3 | PIR_S19363 |
| Structure | Sacch3D_YCL035C |
| Description | Glutaredoxin |
| Function | Glutaredoxin |
| cellular_component | Unknown |
| Process | oxidative stress response |
| Phenotype | Null mutant is viable but sensitive to |
| | oxidative stress. grx1 grx2 null |
| | mutants are viable but lack heat- |
| | stable oxidoreductase activity. |

2. Edges

Information about relationships between biological molecules can be represented by edges of molecular relational graphs. Types of quantitative or qualitative measurements of relationships stored in edges can include but are not limited to the following:

boolean values indicating the presence of association in time, or space, or logical meaning, descriptors of physical or logical states such as “+” representing activation and “−” indicating inhibition, real value measurement of spatial distance such as physical distance between two genes on the chromosome, real value measurement of time or kinetic information such as chemical conversion rate. Euclidean and other distance metrics in feature space to measure logical relationship, correlation coefficient as a statistical metric to measure logical relationship, values of fuzzy set membership function as a metric to measure logical relationship, conditional probability as a measurement of causal relationship, and any combination of these.

Relationships embodied in the disclosed edges can also include physical distances between genes, open reading frames, single nucleotide polymorphisms, expressed sequence tags, sequence tag sites, or a combination thereof; genetic distances between genes, open reading frames, single nucleotide polymorphisms, expressed sequence tags, sequence tag sites, or a combination thereof; protein-protein interactions; protein-nucleic acid interactions; gene expression regulation; protein expression regulation; cellular signal transduction pathways; sequence similarity between genes or proteins; structural similarity between proteins; radiation hybrid mapping distances between genes, open reading frames, single nucleotide polymorphisms, expressed sequence tags, sequence tag sites, or a combination thereof; and metabolic pathways.

The content of an edge can include, for example: (a) labels of two vertices that are connected by the edge; (b) directional labels for the two vertices such as “head” and “tail” indicating the direction of the edge if the relationship is directional between the two biological molecules represented by the two vertices; and (c) an edge weight table which stores properties of the relationship between the two represented biological molecules. The edge weight table of an edge can be organized such that each row of the table contains a label for a relationship property and a value for the corresponding property.

In the disclosed graphs, vertices represent involved biological molecules and edges represent relationships between molecules. Thus the relationship information stored in the edge can include, for example, the identities of participating molecules, the nature of the relationship, and the properties of the relationship. The following illustrations provide examples of creating different types of edges to encapsulate different types of relationship information. As used herein, “relationship” refers to any characterization shared with, linking, correlating, identifying, or otherwise describing any two or more objects (such as biological molecules).

Illustration 4: Defining Edges Representing the Relationship of Protein-protein Interaction between Yeast Protein Molecules

Whole genome-scale study of protein-protein interactions has been carried out for yeast (Uetz et al. (2000)). Out of more than 6,000 proteins, 1,004 yeast proteins were reported to participate in 957 physical interactions with other protein molecules in yeast two-hybrid assays. In order to study large number of protein-protein interactions found in yeast cells, interactions between yeast protein molecules can be represented effectively using edges defined in molecular relational graphs. To define an edge representing a physical interaction between a pair of yeast proteins, vertices representing the two participating protein molecules can be defined first. Once the vertices are defined, an edge can be defined by, for example, the following three components:

(1) Labels of input vertices and output vertices representing the involved protein molecules.

(2) A Boolean variable, DIRECTED, representing whether the edge is directed (thus respecting the input to output designation) or undirected. Since the protein-protein interactions are symmetrical relationships for this example, DIRECTED=FALSE.

(3) An edge weight table in which (property, value) pairs reflecting the properties of relationships are stored. In the simplest form, the table contains a list of (property, value) pairs such as: (assay_system, two hybrid), (assay_method, beta gal), and (strength, 1200).

Assay_method indicates that the lac-Z gene is used as a reporter and β-galactosidase activity mediates the reporter gene activation and the experimental read-out for the assay system. Thus, in this example, the measurement of the strength of interaction is a spectrophotometric measurement of absorption of yeast lysate incubated with β-galactosidase substrate.

To encapsulate the yeast protein-protein interaction data set published by Uetz et al. (2000), 1,004 vertices are created to represent all the involved proteins and 957 edges are created to connect vertices representing the interacting protein pairs.

Illustration 5: Defining Edges Representing Metabolic Pathways in the Cell

In the cell, metabolic molecules such as glucose and amino acids are transformed by various enzymes into different kinds of molecules continuously. These metabolites are either disintegrated into simpler molecules or integrated with other molecules or modified to form more complex molecules. These pathways of molecular transformation can be encapsulated using vertices and edges. To do so, metabolites can be represented by vertices first such that each metabolite is represented by one vertex. Properties of a metabolite such as the name of the chemical compound, the database source of the molecular structure, and cellular localization of the molecule can be stored in the vertex. In the representation of metabolic pathways, an edge can be used to encapsulate a set of metabolic reactions catalyzed by a given enzyme. Thus, an edge connects a pair of vertex groups, one of which represents a group of reaction substrates and the other of which represents a group of reaction products. The definition of an edge for metabolic pathways can comprise, for example, the following information:

(1) A set of labels of input vertices representing reaction substrate molecules;

(2) A set of labels of output vertices representing reaction product molecules;

(3) DIRECTED=TRUE;

(4) An edge weight table can be constructed to contain (property_name, value) pairs of a list of properties including, for example:

(a) Enzyme name: the name of the enzyme that catalyzed the reaction;

(b) Kₘ: the Michaelis-Menton reaction rate coefficient;

(c) Vₘₐₓ: maximum reaction rate under Michaelis-Menton model.

Thus, the edge weight table can encompass information about the identity of the enzyme that catalyzes the reactions and the kinetics that describe the behaviors of the enzyme and the characteristics of the reaction.

Illustration 6: Defining Edges Representing Functional Relationships between Genes of an Organism

Functional relationships between genes are summaries of various relationship information about the functional roles played by these genes. One example of these functional relationships between two genes is that two genes are co-regulated in transcription by the same transcriptional factor. Another example is that protein products of two genes are immediate neighboring elements in a cellular signal transduction pathway. A third example is that protein products of two genes participate in the formation of the same holoenzyme complex. Each edge can encapsulate one elementary type of functional relationship. Multiplexed complex functional relationship representation can be derived using graph operators as discussed below.

To define edges representing functional relationships between two yeast genes, vertices representing the two genes should be defined first. Given the vertices available, an edge can be created to represent each elementary type of functional relationships between two genes. An edge can be constructed by defining a list of information components including, for example:

(1) Labels of input and output vertices representing the two yeast genes—vertex_{—label}1 and vertex_{—label} 2.

(2) Assignment to the variable DIRECTED. For example, for signal transduction pathways, DIRECTED=TRUE.

(3) An edge weight table of properties of the elementary type of functional relationship stored as (property_name, value) pairs. For example, suppose a protein product of gene 2 is a ligand molecule that engages a receptor that is the protein product of gene 1 and the ligand-receptor binding activates the next step of signal transduction cascade. To represent this type of functional relationship, an edge weight table can be constructed to contain (property_name, value) pairs such as:

(Relationship_type, signal transduction)

(Relationship_measurement, K_{d})

(K_{d}, ligand_binding_constant),

where K_{d} is the binding constant which is the measurement of the kinetics of binding process.

B. Graphs

The disclosed vertices and edges make up the disclosed molecular relational graphs. A graph can be constructed to encapsulate information about individual participating biological molecules and information about relationships between them. For example, a molecular relational graph encapsulating gene expression data defines vertices as genes and edges as connections between genes with significantly correlated expression profiles. In another example, a molecular relational graph representing metabolic pathway defines vertices as metabolite molecules, edges as connections between metabolites related to each other by a single biochemical reaction, and edge weights as enzyme that catalyze the reaction between the connected metabolites. As used herein, the terms “graph”, “graphing”, “graphical” are intended to refer to mathematical representations recognized as graphs and are not intended to be limited to be limited to visual depictions of data (although such visual depictions of data are encompassed by the disclosed method).

Possible types of molecular relational graph include but are not limited to the following:

molecular relational graph representing physical mapping of genes, open reading frames, single nucleotide polymorphisms, expressed sequence tags, sequence tag sites, or a combination thereof; molecular relational graph representing genetic mapping of genes, open reading frames, single nucleotide polymorphisms, expressed sequence tags, sequence tag sites, or a combination thereof; molecular relational graph representing radiation-hybrid mapping of genes; molecular relational graph representing orthologous relationships between genes; molecular relational graph representing paralogous relationships between genes; molecular relational graph representing homologous relationships between genes; molecular relational graph representing structural relationships between proteins; molecular relational graph representing gene expression regulation; molecular relational graph representing gene translation regulation; molecular relational graph representing protein-protein interactions; molecular relational graph representing protein-DNA interactions; molecular relational graph representing enzyme functions; molecular relational graph representing chemical metabolism; molecular relational graph representing cellular signal transduction pathways; and molecular relational graph representing functional gene annotation, functional pathways, functional groups, or a combination.

Illustration 7: Construction of a Molecular Relational Graph Representing Gene Expression Data

Microarray technique has been used widely to measure expression patterns for thousands of genes simultaneously. This technique provides a powerful approach for characterizing gene functions in whole-genome scale. In a typical experiment, microarray measurements of gene expression are performed under multiple experimental conditions or at multiple time points of a temporal biological process. The expression profiles of genes across the treatment are then compared and analyzed. The analyses usually consist of a quantification and/or classification of genes into those that display similar expression profiles across the experimental conditions. For example, if the experimental conditions consist of different time-points in a biological process, degree of temporal correlation of expression level for different genes is seen to quantify probability of co-regulation of the genes.

A molecular relational graph representing co-regulation of genes can be constructed by, for example, defining vertices to represent the genes. The method for defining a vertex representing a gene is described in Illustration 3. In this type of graph, an edge connecting a pair of vertices represents the transcriptional co-regulation relationships between a pair of genes represented by the vertex pair. Using methods described in Illustrations 4-6, an edge in this type of graph can include following information items:

(1) Labels of input and output vertices representing the two genes—vertex_{—label}1 and vertex_{—label} 2.

(2) Assignment to variable DIRECTED dependent on experiment.

(3) An edge weight table contains (property_name, value) pairs such as:

(Relationship_type, co-regulation of expression)

(Relationship_measurement, Pearson's correlation coefficient)

(Pearson's correlation coefficient, 0.9).

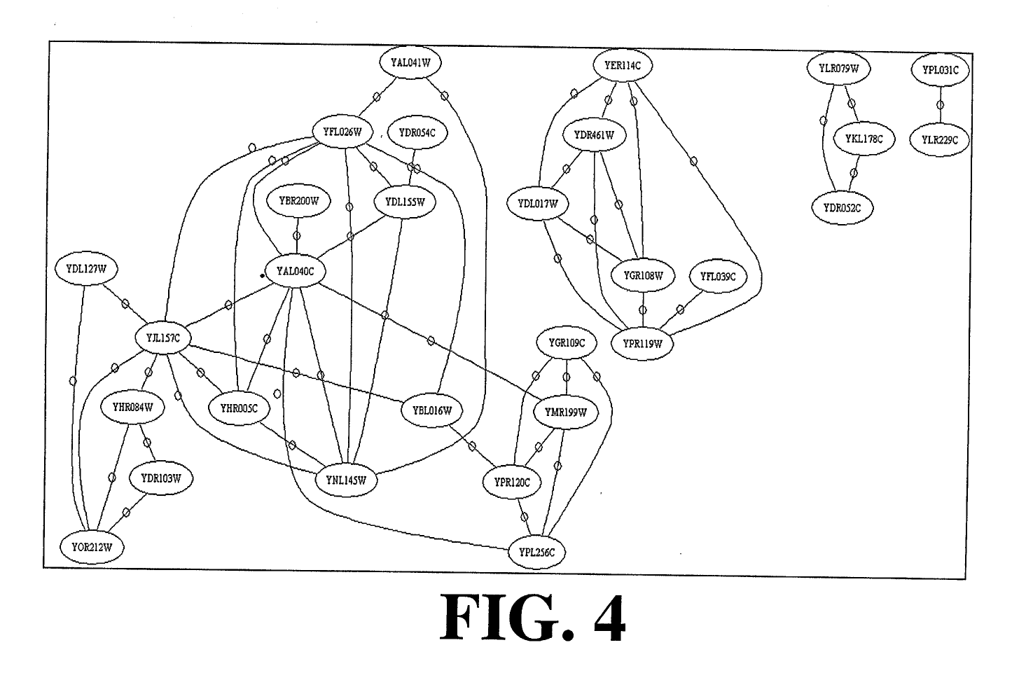

As an example, a molecular relational graph representing microarray hybridization data for gene expression during the yeast cell cycle (Spellman et al. (1998)) was constructed. Pearson's correlation coefficients for the expression profiles of a selected set of gene pairs were computed and used as a metric to measure the co-regulation relationship and stored in the edge weight table for the edges connecting each pair of genes. The resulting molecular relational graph is a completely connected graph in which each vertex is connected to every other vertex. A “threshold” graph-operation can be performed on the edges of the graph to produce a less densely connected graph depicting only the stronger co-regulated relationships. A threshold operator τ(G,crit) removes vertices or edges from graph G, dependent on the criterion set by a conditional statement <crit>. FIG. 4 shows an example where a threshold operator was applied to the co-regulated yeast molecular relational graph using <crit>=if (correlation <0.6). This operation reveals the co-regulation of expression relationships between genes, graded by a degree of confidence. The degree of confidence is determined by the threshold parameter.

Illustration 8: Construction of a Molecular Relational Graph Representing Gene Function Data

A large amount of knowledge about the functions of genes has been accumulated in research and documented in research literature. However, large-scale systematic exploration and comparison of this body of knowledge with research data such as whole genome gene expression profiling data has been hampered by the lack of an annotation system that organizes the knowledge into a form enabling transformation of the literature into computable quantities. To overcome this obstacle, Gene Ontology is the first of such knowledge representation that transforms a large body of knowledge about gene functions into a computable collection of annotations (The Gene Ontology Consortium (2000)). In Gene Ontology (GO), a comprehensive set of descriptions of gene functions is included in the system and each of these descriptions is assigned a unique GO identification number (ID). The descriptions are organized in a way such that descriptions of related functions are connected to each other in a hierarchical tree structure. This tree structure presents the relations between functional descriptions. A gene with known function(s) can be assigned one or more GO IDs. Given functional annotations of genes by GO IDs, the disclosed graphs can be used as an effective approach to reveal functional relationships for a large number of genes.

To create a molecular relational graph based on GO annotations of genes, vertices representing all genes of interests can be defined. Vertex definition is described elsewhere herein (see, for example, Illustration 3). An edge in the graph connects a pair of vertex and encapsulates functional relationship between the two genes represented by the vertex pair. An edge can be defined, for example, by the following:

(1) Labels of input and output vertices representing the two genes—vertex_{—label}1 and vertex_{—label} 2

(2) Assignment to variable DIRECTED depending on the GO function.

(3) An edge weight table of properties of the functional relationship stored as (property_name, value) pairs. As an example, protein product of gene 2 is a transcriptional factor that activates the transcription of gene 1. To represent this type of functional relationship, an edge weight table can be constructed to contain (property_name, value) pairs such as:

(Relationship_type, transcriptional regulation)

(Relationship_measurement, K)

(K, <transcriptional activation_rate_constant>).

K is a rate constant used to characterize the kinetics of transcriptional activation process.

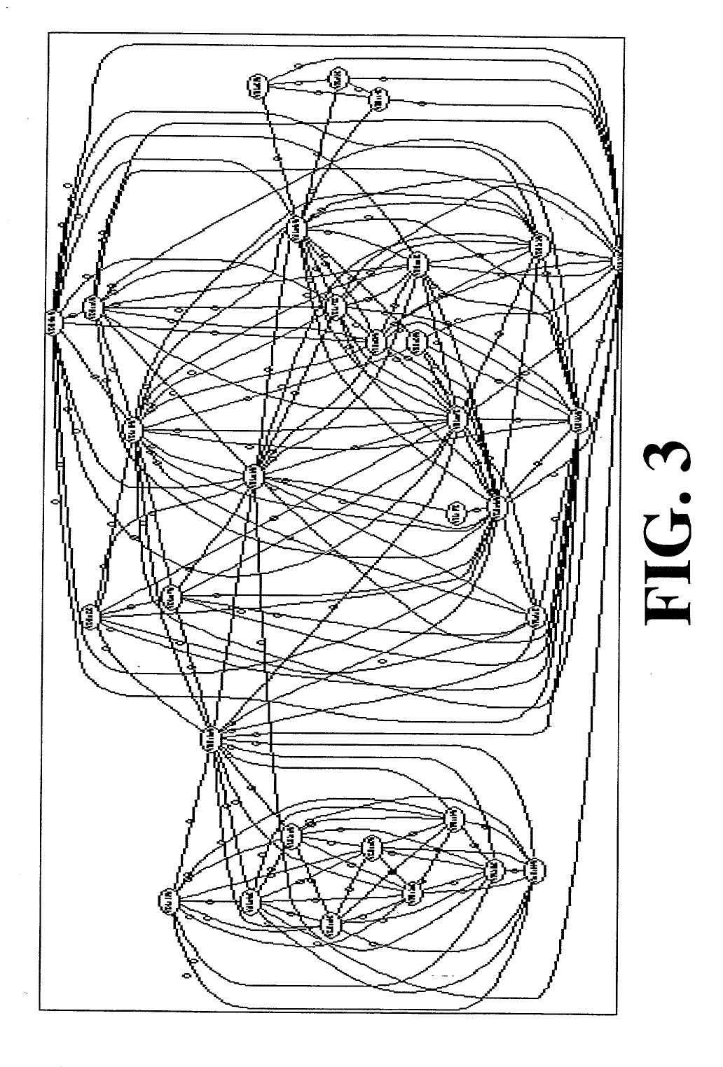

When multiple functional relationships happen between a pair of genes, a graph can be constructed for each functional type and merged with the AND graph operator as described elsewhere herein. FIG. 3 shows an example of using Gene Ontology (GO) functional annotations for a selected set of yeast genes. Yeast GO functional annotation data were imported from the Web site of Gene Ontology Consortium (http://www.geneontology.org/) and used to define edges between the subset of genes. Connected genes share the same unique GO functional identifier. The graph in FIG. 3 clearly shows known functional relationships for a subset of yeast genes. More importantly, from an inspection of the molecular relational graph, one can deduce higher-order functional gene relationships not previously characterized.

C. Operators

Operators used in the disclosed method (referred to herein as operators, molecular relational graphing operators, or gene-graph operators) are any operation or function that can be used to manipulate, transform, combine, split, separate, filter, or otherwise alter one or more graphs to produce one or more product graphs. Operators that can be used on the disclosed graphs can manipulate the graphs as objects, much as mathematical operators manipulate numbers. Like mathematical operators, molecular relational graphing operators and gene-graph operators allow direct manipulation of graphs using graph operations such as difference, addition, and intersection. Operators can be recursive. The disclosed method is not limited to the operators described herein. Numerous graph operators and graph manipulation procedures are known and can be used in the disclosed method. As used herein, “operation” refers to the use of one or more operators on one or more graphs. The disclosed graphs are generally mathematical constructs describing biological molecules that can be manipulated, transformed, combined, split, filtered or otherwise altered using any relevant mathematical operator.

Operators are defined for computing molecular biological information using graphs defined above as operand(s). Rules can be defined for construction of biologically meaningful computations. Two or more graphs can be manipulated to yield a third graph. Such manipulations allow synthesis of disparate biological information encapsulated in different molecular relational graphs.

Graph operators include unary operators, binary operators, and n-nary operators. Useful unary operators include, for example:

“Threshold edges” which deletes all edges below or above a particular range of edge weights;

“Threshold vertices” which deletes all vertices below or above a particular range of vertex parameters;

“Subset” which is inclusive of only certain edges or vertices (if applied to vertices, inapplicable edges are also deleted);

“Split” which divides one graph into two graphs;

“Convert graph” which converts a graph from one type to another so that graphs of different types can be comparable.

Useful binary and n-nary operators include:

“And” which, given n graphs, finds the common subset of vertices and edges and outputs the graph containing only the common vertices and edges;

“Or” which, given n graphs, finds the union of all vertices and edges and outputs the graph containing the union;

“Addition” which grafts two different graphs A and B together if the two different graphs have common vertices;

“Subtraction” which deletes from a third graph X any vertices common to a first graph A and a second graph B;

“Filtration” which compares and generates a graph X wherein all edges (vertices) in compared graphs A, B, etc. that are not also in X are deleted;

“Consensus” which provides an X% consensus graph of graphs A, B, etc. which is defined as a graph consisting of all vertices and edges present in X% or more of the graphs, A, B, etc.

Useful Vertex and Edge operations used in the present invention include:

“Delete” which deletes a vertex (edge);

“Add” which adds a vertex (edge);

“Combine” which combines two or more vertices into one retaining the edges to all other vertices or combines two or more edges into a hyper-edge;

“Examine vertex” which shows information contained in a vertex such as its label (gene name), mapping location, amino-acid composition, and can show, for example, information obtained through an outside database via a URL linkage;

“Examine edge” shows information contained in an edge such as activation/repression nature of the gene relationship, catalytic rate constant of the enzyme reaction, and binding affinity between two protein molecules.

Operators can be depicted using symbols. This can aid in combining operators into sets and series, and in constructing complex operators. An example of a system of operator symbols and their use is described below. Additional operators are also provided below.

1. Unary Operators (Λ)

Threshold edges (Λ₁): Delete all edges below (or above) a particular range of edge weights.

Threshold vertices (Λ₂): Delete all vertices below (or above) a particular range of vertex parameters.

Subset (Λ₃): Inclusive of only certain edges or vertices. If applied to vertices, irrelevant edges are also excluded.

Split (Λ₄): Divide one graph into two graphs.

Find topological sorting for a set of vertices (Λ₅): Find a linear order for a set of vertices in a graph such that any graph traversal path constructed from the sorting preserves the original order of vertex-to-vertex connection in the graph.

Find shortest path from vertex A to B (Λ₆): Identify a path starting from vertex A and ending at vertex B. The number (if un-weighted graph) or the sum of weights (if weighted graph) of edges involved in the path is minimum compared to any other possible path.

Find shortest path between each pair of vertices (Λ₇): Identify a path for each pair of vertices. The path connects two vertices in the pair and the number (if unweighted graph) or the sum of weights (if weighted graph) of edges involved in the path is minimum compared to any other possible path.

Find transitive closure (Λ₈): Construct for a graph a vertex reachability matrix in which the value of an element located at i-th row and j-th column represents vertex j is reachable from vertex i if the value equals to 1 or else 0.

Find articulation points (Λ₉): Traverse the graph and identify all vertices the deletion of which splits the graph into two or more substructures. An articulation point usually represents a junction linking multiple pathways or subsystems, for example, a gene that participates in multiple biological processes.

Find strongly connected components (Λ₁₀): Traverse the graph and identify all subsets of vertices whose connections to vertices within the same subset are much denser than are connections to vertices outside the subset. A subset usually reflects a relatively complete and independent functional group of genes participating in a single biological process.

Find minimum-weight spanning tree (Λ₁₁): Construct a tree from a graph so that the tree contains all the vertices in the graph and the sum of weights of all edges in the tree is minimum. A tree is a graph with properties: a) any two vertices are connected by precisely one path; b) no vertex can reach itself through a path including zero or more edges and/or vertices.

Find maximum-weight spanning tree (Λ₁₂): Construct a tree from a graph so that the tree contains all the vertices in the graph and the sum of weights of all edges in the tree is maximal.

Find fundamental circuits (Λ₁₃): Find a set of circuits in a graph so that any circuit present in the graph can be derived from a ring-sum of a combination of elements in the set. A ring-sum of two graphs G₁=(V₁, E₁) and G₂=(V₂, E₂) is the graph ((V₁∪V₂), ((E₁∪E₂)−(E₁∩E₂)).

Find fundamental cut-sets (Λ₁₄): Find a set of cut-sets in a graph so that any cut-set of the graph can be derived from a ring-sum of a combination of elements in the set. A cut-set of a connected graph or component is a set of edges whose removal will disconnects the graph or colmponent.

Find the capacity of a cut-set (Λ₁₅): Calculate the flow capacity of a cut-set of a graph. Given a vertex, x, as the source and another vertex, y, as the sink of a network N, a flow for N associates a non-negative integer f(u, v) with each edge (u, v) of N, such that for all vertices v, other than x or y:

$${\sum }_{u} f ( u , v ) = {\sum }_{u} f ( v , u ) .$$

An edge capacity c(u, v) is defined as the maximum of f(u, v) for the corresponding edge. A cut-set of a graph (V, E) partitions vertices into two sets (P, \overline{P}) such that P∩\overline{P}=Ø and P∪\overline{P}=V. The capacity of the cut-set is then defined as

$${\sum }_{{u ∈ \underline{P}}_{v ∈ P}} c ( u , v ) .$$

Condense graph (Λ₁₆): Collapse each component in a graph into a hyper-vertex and replace edges incident to and from the component with edges incident to and from the hyper-vertex.

Convert graph (Λ₁₇): Transform a graph from one type to another so that graphs from different sources can be compared.

Find connected components (Λ₁₈): Identify all connected components in a graph.

2. Binary and n-nary Operators (Ξ)

AND (Ξ₁): Given n graphs, find the common subset of vertices and edges. Output the graph containing only the common vertices and edges.

OR (Ξ₂): Given n graphs, find all vertices and edges. Output the graph containing all vertices and edges present in either graph.

Addition (Ξ₃): If two different graphs have common vertices, merge the two graphs.

Subtraction (Ξ₄): Given graph A and graph B with common vertices, subtraction of graph B from graph A is the operation that deletes from graph A all vertices common to graph B, thus producing graph C, such that C=A−B.

Filtration (Ξ₅): A filtration of graphs by some graph X is the process of deleting all edges (or vertices) in each graph that are not also present in graph X.

Consensus (Ξ₆): An X% consensus graph is the graph consisting of all vertices and edges present in X% or more of the graphs on which the operation is performed.

Isomorphism (Ξ₇): Given graphs G₁=(V₁, E₁) and G₂=(V₂, E₂), find a graph G₃=(V₃, E₃) such that: a) there is a bijection f₁: V₁^{S}→V₃ such that {f₁(x), f₂(y)}εE₃ if and only if {x, y}εE₁; b) there is a bijection f₂: V₂^{S}→V₃ such that {f₂(x), f₂(y)}εE₃ if and only if {x, y}εE₂ where V₁^{S} and V₂^{S} are subsets of V₁ and V₂ respectively. A bijection is a function f: A→B if it is both an injection (one-to-one) and a surjection (the reverse is also one-to-one)(Ore, Theory of Graphs, American Mathematical Society, Providence, R.I. (1962)).

3. Vertex and Edge Operators (Ψ)

Delete (Ψ₁): Remove a vertex (or edge).

Add (Ψ₂): Insert a vertex (or edge).

Union (Ψ₃): Combine two or more vertices into one vertex retaining the previously existing edges to all other vertices. Combine two or more edges into a hyper-edge.

Disassemble (Ψ₄): Disassemble a hyper-vertex and/or a hyper-edge formed as a result of Union operation into original set of vertices and/or edges.

Examine vertex (Ψ₅): Show information contained in a vertex, such as its label, gene name, mapping location, amino-acid composition, and URL to external databases.

Examine edge (Ψ₆): Show information contained in an edge such as activation/repression nature of the gene relationship, catalytic rate constant of the enzyme reaction, or binding affinity between two protein molecules.

4. Rules

Any computation on molecular relational graphs using molecular relational graph operators can be constructed by following rules. The following rules are examples of useful rules. In the rule definitions, G₁, G_{2,} G₃, . . . Gₙ and G each represents a different molecular relational graph and Ø is an empty set.

(i) Rules of Modifiers

Rules of modifiers can define the syntax for using modifier-style operators, Λ and Ψ. An operator of this type operates on a single input graph:

Λᵢ(G₁)=G₂, where i={1, 2, 3, 6, 7, 11, 12, 16, 17}

Λᵢ(G)=S, where S={G₁, G₂, . . . } and i={4, 10, 13, 14, 18}

Λᵢ(G)=S, where S={V₁, V₂, . . . } and i={5, 9}

Λᵢ(G)=M, where M is a reachability matrix and i={8}

Λᵢ(G)=C, where CεR and i={15}

Ψ(G, S)=G where S={V1, V2, . . . } and i={8}

(ii) Rules of Binary Operation

Rules of binary operation can define the syntax for using binary operators, which take two input graphs and produce an output graph:

G₁ΞᵢG₂=G₃, where i={1, 2, 3, 4, 5, 7}

(iii) Rules of n-nary Operation

Rules of n-nary operation can define the syntax for using n-nary operators, which take more than two graphs as input and produce different types of output:

Ξᵢ(G₁, G₂, G₃, . . . , Gₙ)=G, where i={1, 2}

Ξᵢ(S, G)=S′, where S={G₁, G₂, G₃, . . . , G_{n}, S′={G}₁′, G₂′, G₃′, . . . , Gₙ′} and i={5}

Ξᵢ(X%, G₁, G₂, G₃, . . . , Gₙ)=G, where X% εR and i={6}

(iv) Empty Graph Laws

Empty graph laws can define the result of computation for various operators when an empty set, Ø, is involved in the input:

Λᵢ(Ø)=Ø, where i={1, 2, 3, 4, 5, 6, 7, 9, 10, 11, 12, 13, 14, 16, 17}

Λᵢ(Ø)=M, where M is a reachability matrix with all elements equals to 0 and i={8}

Λᵢ(Ø)=0, where i={15}

Ψ(Ø, S)=Ø

Ψ(G, Ø)=G

GΞᵢØ=Ø, where i={1, 6, 7}

GΞᵢØ=G, where i={2, 3, 4, 5}

Ξᵢ(Ø, G₂, G₃, . . . , Gₙ)=Ø, where i={1}

Ξᵢ(Ø, G₂, G₃, . . . , Gₙ)=i (G₂, G₃, . . . , Gₙ), where i={2}

Ξᵢ(S, Ø)=S, where S={G₁, G₂, G₃, . . . , Gₙ} and i={1}

Ξᵢ(C, Ø, G₂, G₃, . . . , Gₙ)=Ø, where CεR and i={6}

(v) Idempotency Laws

Idempotency laws can define the result of computation for binary and n-nary operators when identical graphs are taken as the input:

GΞᵢG=G, where i={1, 2, 3, 7}

GΞᵢG=Ø, where i={4, 5}

Ξᵢ(G, G, G, . . . , G)=G, where i={1, 2, 3, 7}

Ξᵢ(G, G, G, . . . , G)=Ø, where i={5}

(vi) Commutative Laws

Communitive laws state that, in consecutive binary operations, operands involved can exchange positions freely without affecting the end result:

G₁ΞᵢG₂=G₂ΞᵢG₁, where i={1, 2, 3, 4, 7}

(vii) Associative Laws

Associative laws state that the order of a sequence of operations performed by binary or n-nary operators can be rearranged without affecting the end result:

(G₁ΞᵢG₂)ΞᵢG₃=G₁Ξᵢ(G₂ΞᵢG₃), where i={1, 2, 3, 4, 5, 6, 7}

(viii) Distributive Laws

Distributive laws state that the product of a first binary or n-nary operation on the product of a second binary or n-nary operation on some objects will yield the same result as the second binary or n-nary operation on the products of the first binary or n-nary operation on each of the objects:

G₁Ξᵢ(G₂ΞⱼG₃)=(G₁ΞᵢG₂)Ξⱼ(G₁ΞᵢG3), where i={1, 4, 5, 6, 7}, j={1, 2, 3, 4, 6, 7}, and i ≠j

Λᵢ(G₁ΞⱼG₂)=(Λᵢ(G₁))Ξⱼ(Λᵢ(G₂)), where i={1, 2, 3, 6, 7, 11, 12, 16, 17}, j={1, 2, 3, 4, 6, 7}

5. Methods for Assimilating Disparate Molecular Biological Data

(i) Integration of Disparate Data Sets

Two or more non-overlapping data sets, {G₁, G₂, G₃, . . . , Gₙ}, can be synthesized into a single data set, G:

G=Ξ₂(G₁, G₂, G₃, . . . , Gₙ) or G=G₁Ξ₂G₂

Two or more overlapping data sets, {G₁, G₂, G₃, . . . , Gₙ}, can be synthesized into a single one, G:

G=Ξ₃(G₁, G₂, G₃, . . . , Gₙ) or G=G₁Ξ₃G₂

(ii) Filtration of a Data Set Using Another Data Set

Subtraction of data found in one data set, G₂, from another data set G₁ and yield a third data set, G₁′:

G₁′=G₁Ξ₄G₂

Filtering out consensus data between one data set, G1, and another data set, G2, from data set G1 and yield a third data set, G₁′:

G₁′=G₁Ξ₅(G₁Ξ₁G₂)

(iii) Identification of Consensus Data From Disparate Data Sets

Identification of consensus data, G, between two data sets, G₁ and G₂, without having to preserve the relationships between biological molecules in original data sets:

G=G₁Ξ₁G₂

Identification of consensus data, G, between two data sets, G₁ and G₂, such that the original relationships between biological molecules are preserved in the resulting data set:

G=G₁Ξ₆G₂

Identification of consensus data, G, among many data sets, G₁, G₂, G₃, . . . , Gₙ, such that the consensus data appears in more that X% of total number of data sets:

G=Ξ₆(X%, G₁, G₂, G₃, . . . , Gₙ)

(iv) Identification of Unique Data for Individual Disparate Data Sets

Identification of data, (G_{1, unique}, G_{2, unique}, G_{3, unique}, . . . , G_{n, unique},), unique for individual data sets, (G₁, G₂, G₃, . . . , Gₙ)—method (I):

G_{consensus}=Ξ₁(G₁, G₂, G₃, . . . , Gₙ)

G_{1, unique}=G₁Ξ₄G_{consensus}

G_{2, unique}=G₂Ξ₄G_{consensus}

G_{3, unique}=G₃Ξ₄G_{consensus}

. . .

G_{n, unique}=GₙΞ₄G_{consensus}

Identification of data, (G_{1, unique}, G_{2, unique}, G_{3, unique}, . . . , G_{n, unique},), unique for individual data sets, (G₁, G₂, G₃, . . . , Gₙ)—method (II):

G_{consensus}=(. . . ((G₁Ξ₇G₂)Ξ₇G_{3 )Ξ}₇. . . )Ξ₇Gₙ

G_{1, unique}=G₁Ξ₄G_{consensus}

G_{2, unique}=G₂Ξ₄G_{consensus}

G_{3, unique}=G₃Ξ₄G_{consensus}

. . .

G_{n, unique}=GₙΞ₄G_{consensus}

(v) Identification of Common Biological Pathways Revealed by Two Different Data Sets

To find a set of biological pathways, S, that are revealed in both data sets, G1 and G2, one identifies strongly connected components in both graphs first. Then condenses those components into hyper-vertices. An isomorphic sub-graph, G, of G₁ and G₂ is subsequently identified. Pathways can then be isolated from G and stored in S:

G=(Λ₁₆(G₁, Λ₁₀(G₁)))Ξ₇(Λ₁₆(G₂, Λ₁₀(G₂)))

S=Λ₁₈(G), where S is a set of graphs, each of which represents a pathway common to both data set G₁ and G₂

(vi) Identification of Biological Molecules Critical for Multiple Biological Pathways

To identify biological molecules critical for multiple biological pathways (G₁, G₂, G₃, . . . , Gₙ), one identifies articulation points in each graphs first (V₁, V₂, V₃, . . . , Vₙ) and subsequently find an intersection set, V, of vertex set (V₁, V₂, V₃, . . . , Vₙ):

V₁=Λ₉(G₁)

V₂=Λ₉(G₂)

V₃=Λ₉(G₃)

. . .

Vₙ=Λ₉(Gₙ)

V=V₁∩V₂∩V₃∩. . . ∩Vₙ

6. Ancillary Functions

“Find articulation points” which traverses the graph and identifies all the vertices that, when deleted, can split graph into two or more substructures; an articulation point usually represents the cross-linking point among multiple pathways or subsystems, for example, a gene functions in multiple biological processes.

“Find strongly connected components” which traverses the graph and identifies all subsets of vertices whose connections to vertices within the same subset is much denser than to the outside vertices; a subset usually reflects a relatively complete and independent functional group of genes participating in a single biological process.

7. Assimilating Disparate Molecular Biological Data

Large-scale and high throughput biological experiments such as whole genome gene expression and protein translation profiling produce disparate data of large size. The complexity of the relationship information embedded in these data made analysis difficult using prior methods. Moreover, these data contain different types of relationship information depending on the design and the purpose of the experiments generating the data. The heterogeneity of these data presented a serious challenge to the integration of information using prior methods. The disclosed method is particularly apt for handling the complexity and heterogeneity of data and is thus capable of facilitating the integration and understanding of large-size heterogeneous biological data. Two examples of the application of the disclosed method to complex data are described below and illustrate these capabilities.

Illustration 9: Integration of Gene Expression Data with Gene Ontology Data

Microarray gene expression data contain information about expression profiles for a large number of genes. From this type of data, gene functions can be inferred by comparing expression profiles between genes. Genes having similar expression profiles are considered to have high probability of being co-regulated by the same transcriptional control mechanism and thus may contribute to the creation of the same phenotype. While analyses of newly generated data using state-of-the-art technology give tremendous insights into gene functions, discoveries made in previous research also accumulate a large body of knowledge that needs to be merged together with current progress in order to facilitate the formation of a comprehensive understanding of gene functions. One good example of such previously accumulated knowledge is Gene Ontology annotations. Integration of gene co-regulation information with functional annotation of genes is needed to produce a comparison of these two bodies of information. This integration can be done by the synthesis of information represented by the disclosed methods. Gene expression data (Spellman et al. (1998)) and GO annotation for yeast genes were chosen to illustrate the ability of graph-operators to derived integrated representation of heterogeneous information.

A graph of gene expression profiles was generated from the data as described in Illustration 7. In this graph, relationships of expression co-regulation between genes are captured by the edges. A second molecular relational graph representing GO annotation of genes is generated as described in Illustration 8. To simplify the computation, the graph representing GO functional relationships was created as an unweighted graph by omitting the step of creating an edge weight table. Since the graph of GO functional relationships was an unweighted graph, while the graph of gene expression was a weighted graph in which the edge weights were the correlation coefficients, the unary operator “convert”, c(G, t₁, t₂), was used to transform a graph (G) from one type (t₁) to another (t₂), so that graphs from different sources can be compared. Thus the operator c(G, t₁, t₂), where t₁=WEIGHTED and t₂=UNWEIGHTED, transformed the weighted graph shown in FIG. 4 to an unweighted graph.

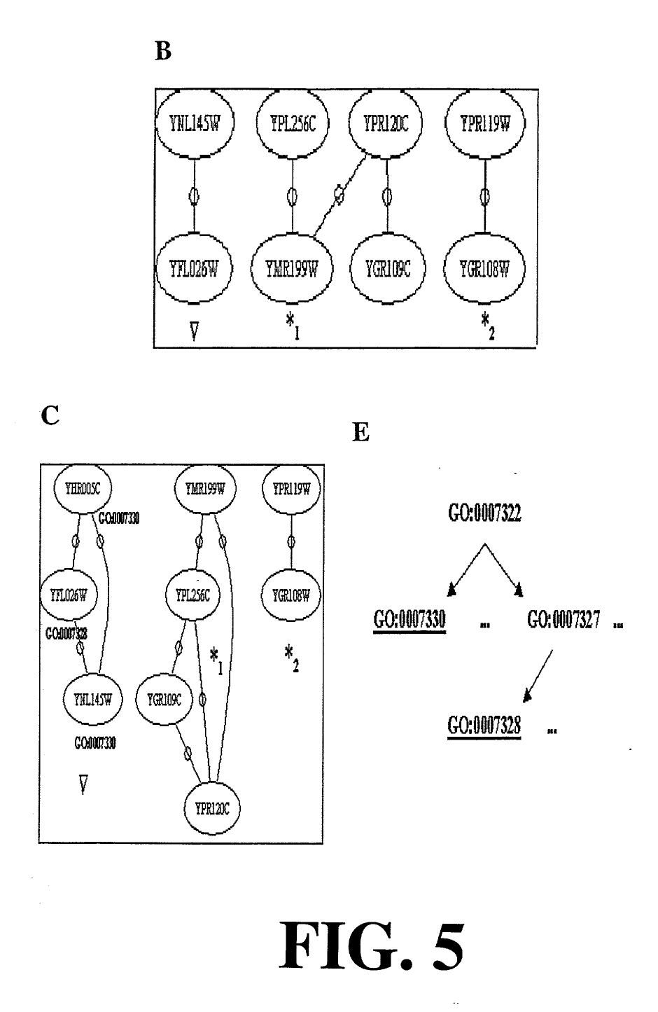

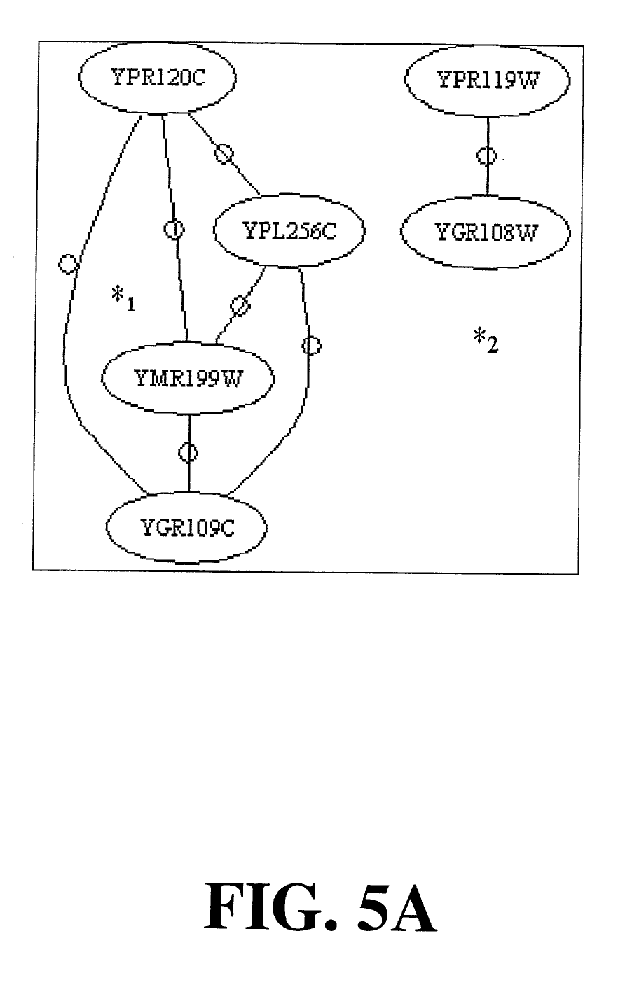

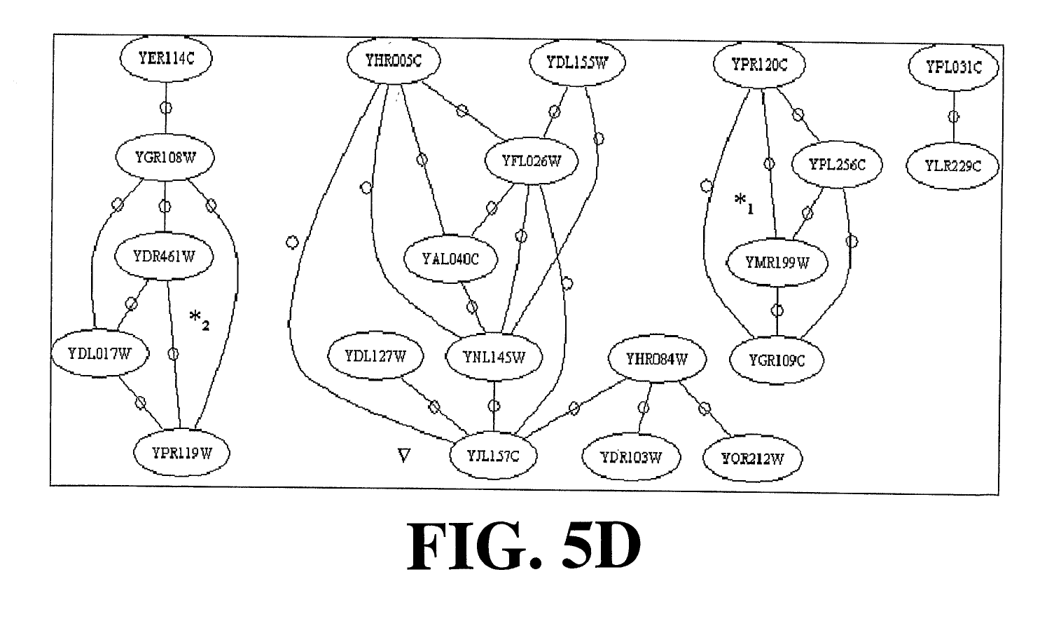

To integrate the two types of information, the graph of the complete set of GO functional relationships (not shown) and a graph of gene expression data (FIG. 4) were input to the graph operator “AND”. The binary operator “AND” synthesizes information from two or more graphs by finding the subset of common edges and vertices. The resulting consensus information is shown in FIG. 5A. Because only a subset of the 6,000+ yeast genes is used to generate FIG. 4, the results shown in FIG. 5A are for illustrative purposes only, and do not represent an exhaustive survey. FIG. 5A shows two connected component structures representing two distinct sets of genes. These sets represent those genes whose GO functional relationships are concordant with their expression pattern relationships.

Illustration 10: Exploratory Thresholding of Gene Expression Data

In a weighted graph representing co-expression relationships of genes, every vertex can be connected with all other vertices through edges. The edge-weights, correlation coefficients, for this type of graph quantifies the degree of co-expression. The quantitative information in the correlation coefficients can be used to generate a coarser representation graph showing only those relations with high confidence. For this purpose, the edge filtering operation on molecular relational graphs can be performed by the “threshold” operator τ(G, crit), which removes vertices or edges from graph G, dependent on the criterion set by a conditional statement <crit>.

As an example of exploratory thresholding applied to gene expression graphs, threshold operations were performed on the graph shown in FIG. 4 to determine whether stronger correlations in gene expression are related to functional relationships. That is, it was asked whether the structure shown in FIG. 5A can be recovered from the graph shown in FIG. 4 alone by including only the strongest co-expression relationships. In fact, both of the connected graph components seen in FIG. 5A appear in gene expression graphs thresholded at 0.9 (FIG. 5B), 0.8 (FIG. 5C), and 0.7 (FIG. 5D). Higher-stringency thresholding produces fewer gene-relationship structures in the expression data, but more of the structures produced are supported by the GO functional annotations. This suggests a quantitative relationship between concordant expression of genes and their functional interaction. In addition, FIG. 5 shows that the expression data also imply some gene relationships (marked by ∇ in FIGS. 5B, 5C, and 5D) which are not apparent in the GO annotation graph (FIG. 3). Careful examination shows that a higher-order relationship documented in the GO tree can account for these expression relationships (FIG. 5E). This exercise demonstrates how a novel functional inference could be made through the power of integrative analysis using the disclosed method. Operations used to generate FIG. 5 are summarized in the Table 4.

TABLE 4
Operations used to generate the molecular relational graphs
shown in FIG 5.

| | | | Resulting |
|---|---|---|---|
| Graph A | Graph B | Operator | Graph |
| GO graph | Gene expression graph | AND | |
| | Gene expression graph | τ(G, crit) | |
| | | <crit> = | |
| | | if (correlation < 0.9) | |
| | Gene expression graph | τ(G, crit) | |
| | | <crit> = | |
| | | if (correlation < 0.8) | |
| | Gene expression graph | τ(G, crit) | |
| | | <crit> = | |
| | | if (correlation < 0.7) | |

D. Implementation

In one embodiment, a software program for GGO can be developed using the JAVA programming language. This program has two principal features, the first being the implementation of molecular relational graph objects and the ability to persist to a local database, and the second being implementation of the set of operators that can be performed on the gene-graphs. This software performs the task of integrating the data from microarray gene expression analysis, Gene Ontology annotation, and protein-protein interaction analysis into a GGO data model functionalities for pathway analysis, critical gene identification, gene-action subsystem identification, and pathway comparison. Since the molecular relational graphing model is best illustrated using a graphical approach, in a preferred embodiment, the software provides visualization essential for the demonstration of the data resulting from the computation using GGO data model. In a preferred embodiment, the visualization software is based on three development resources: JAVA 2D and JAVA3D API libraries developed by SUN MICROSYSTEM which provide classes for writing two- and three-dimensional graphics applications; Open source software Graphviz developed by AT&T Laboratory (www.research.att.com/sw/tools/graphviz/) which is a set of tools for construction and geometric presentation of graphs and networks with a publicly available source code allowing use to build complex visualization functionality; and commercially available graphics API libraries developed by Advanced Visual Systems.

Standard analysis techniques can be integrated into this analysis platform by incorporating standard commercial software packages. This allows the system to use many analysis features, such as clustering analysis, from other packages for preliminary data processing. The resulting data is then ported into the molecular relational graphing model for high-level analysis.

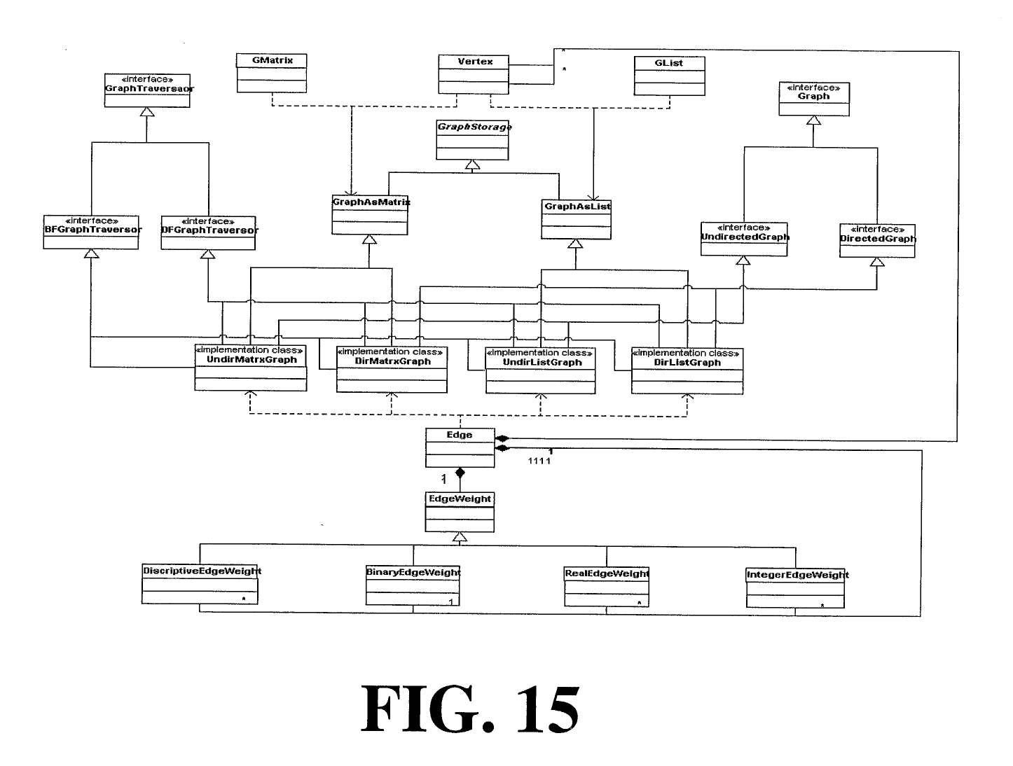

An Unified Modeling Language entity diagram of GGO objects employed in the design of this software is depicted in FIG. 15.

The analysis capability of the molecular relational graphing data model is exemplified in part by the following conversion of genomic information into graph structure. Software has been developed to convert genomic information to graph structure. Various graph operators have also been implemented for the MRG model, including, but not limited to, add and delete vertex, add and delete edge, threshold edges, subset, graph AND, and graph OR. Using these programs, data from microarray gene expression assays, protein-protein interaction assays, and Gene Ontology functional annotation have been encoded into graph structures. Further, a set of graph visualization tools have been incorporated into the program.

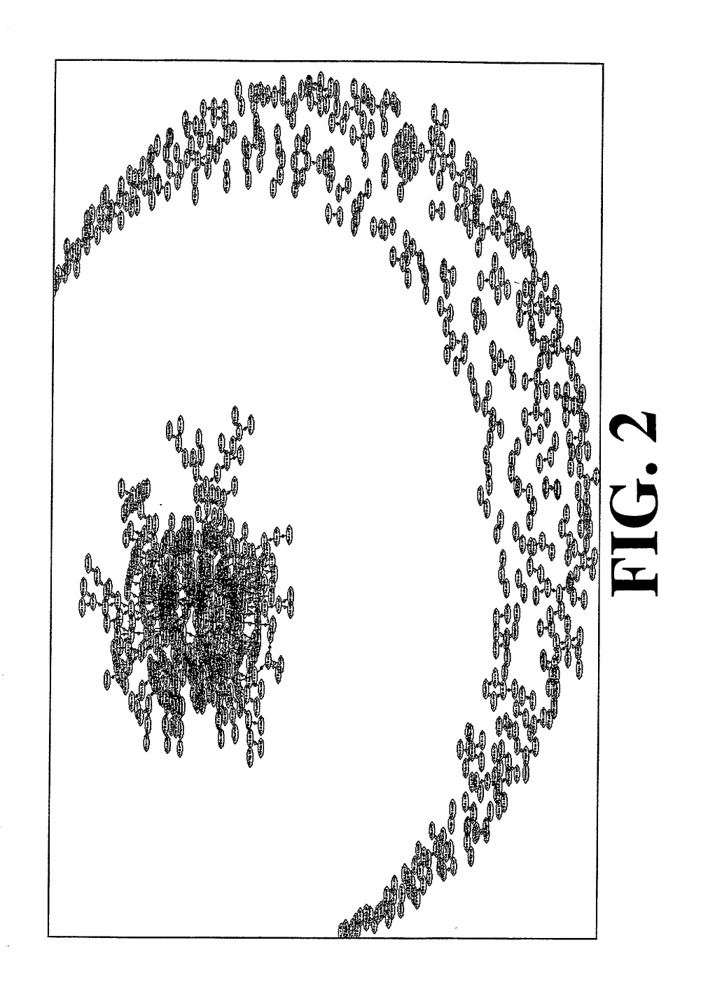

Exemplary results are shown in FIGS. 2 through 5. In FIG. 2, data were imported from the analysis of the yeast (Saccharomyces cerevisiae) genome and encoded into gene-graphs. In this application, 1,004 genes and 957 protein-protein interactions documented in Uetz et al. (2000) were graphed. The resulting visualization reveals structural complexities such as the subset of strongly connected components seen in the middle of FIG. 2.

Similarly, FIG. 3 shows a graphical representation of functional relationships found in the Gene Ontology (GO) database for a selected set of yeast genes. The resulting graph encapsulates previous knowledge of the function of these genes. A comprehensive view of the functional relationships among the genes is clearly revealed by the gene-graph. Importantly, the gene-graph representation reveals higher-order functional gene relationships not previously characterized.

Quantitative relational data such as correlations can also be represented as a graph structure. As an example of this, microarray hybridization data were analyzed for gene expression during the yeast cell cycle (Spellman et al. (1998)). The expression profile correlations of all gene pairs were computed and used as a metric to define the edge weight for the edges connecting each pair of vertices, here defined as genes. The gene-graph thus generated encapsulates the relationships of the gene expression profiles. The unary operation “thresholding” converts quantitative relational information into more intuitive qualitative information with a tunable parameter. A threshold operation on the graph of gene expression was performed. A threshold of 0.4 was chosen, where a value of 0 corresponds to no correlation, and a value of 1 to complete correlation. In this threshold operation, edges were deleted if their weights were greater than or equal to 0.4. The resulting graph is shown in FIG. 4. This operation reveals the expression relationship between genes, graded by the degree of confidence as measured by a quantitative parameter.

Information from two or more kinds of gene-graph can be synthesized using the graph operation AND. FIG. 5 presents such a synthesis of information between the functional relationship indicated by the GO gene-graph and the Spellman et al. expression study. The AND operator was used with different threshold operators on the expression graph to demonstrate how graph operators can be combined to yield a flexible set of information syntheses. FIG. 5A, shows the results of an AND operation between the GO annotation graph and gene expression graph thresholded at the 0.4 level. The result produces two connected component structures representing two distinct sets of genes whose functional relationships are concordant with their expression pattern relationships. Both structures appear in expression gene-graphs thresholded at 0.1 (FIG. 5B), 0.2 (FIG. 5C), and 0.3 (FIG. 5D). Higher-stringency thresholding produces fewer gene-relationship structures in the expression data, but more of the produced structures are in conformity with the GO data. This indicates a quantitative relationship between concordant expression of genes and their functional interaction. FIG. 5 shows a relationship between genes implied by the expression data that is not apparent in the GO data (marked by ∇). However, careful examination shows that a second order interaction documented in the GO accounts for the expression relationship (FIG. 5E). This is a novel discovery mediated by the power of integrative analysis from the GGO model of the present invention.

Accordingly, as demonstrated herein, gene-graph analysis provides a powerful tool for the analysis of large genomic data sets and the discovery of novel gene relationships, as well as for the corroboration of relational data by drawing consensus from disparate sources of information. Further enrichment of the algorithmic operations on the gene-graph by adding new theoretical and heuristic components can greatly expand the potential of this analytical technique and transform it into a significant discovery tool for genome-scale data analysis.

The disclosed method can be produced and used at varying levels from software components to integrated packages with user-interface which allows a wide range of application. Different graph manipulation tools can be implemented, for example, as reusable JAVA components. In addition, GGO software may be readily interfaced with other software packages, such as common statistical packages. A useful component of the integrative data analysis package of the disclosed method is to enable preliminary data processing, such as cluster analysis. Common statistical packages could be used to provide such analyses. Thus, all or part of the disclosed method can be implemented as macros and routines to interface statistical analysis packages such as SAS, SPSS, SPLUS using the GGO data model.

Software design process for implementing the disclosed method preferably can employ the object-oriented notation, UML (Unified Modeling Language, Booch et al.), to document requirements, classes, class behavior, and class dependencies of molecular relational graphing software. A UML entity diagram of a selection of molecular relational graphing objects is shown in FIG. 15. In order to capture the architectural design of the molecular relational graphing software, user interface story-boards, use case diagrams, sequence diagrams, and class hierarchy diagrams can be developed.

E. Embodiments

The disclosed method, structures, and compositions can be further understood with the following descriptions of some of their forms and embodiments.

One embodiment of the disclosed method is a computer-implemented method for performing an operation upon one or more graphs, wherein each graph can represent a set of relationships between a set of biological molecules, wherein each graph can comprise vertices representing the biological molecules and edges representing the relationships between the biological molecules, where the method comprises performing one or more operations on the one or more graphs to produce one or more product graphs.

Another embodiment of the disclosed method is a computer-implemented method for performing an operation upon a graph, where the graph can represent relationships between biological molecules and can have vertices representing the molecules and edges representing the relationships, where the method comprises identifying a subset of zero or more of the edges, identifying a subset of zero or more of the vertices, and performing a unary operation upon the identified subset of edges and vertices to produce a product graph. As used herein, “identifying a subset” of vertices and/or edges refers to selecting, using any desired criteria, those vertices and/or edges in a set of vertices, set of edges, and/or graph(s) having or lacking one or more of the desired criteria features.

Another embodiment of the disclosed method is a computer-implemented method for representing relationships between biological molecules using one or more graphs each having vertices and edges, where the method comprises representing a set of biological molecules, wherein each molecule can be represented by a vertex of the graph, and representing a set of relationships between the biological molecules, wherein each relationship can be represented by an edge of the graph, wherein the edge connects two vertices, wherein the graph can be produced by performing one or more operations on one or more input graphs to produce the one or more graphs. The disclosed graphs represent relationships between biological molecules.

One embodiment of the disclosed composition is a computer program product for performing an operation upon one or more graphs, wherein each graph can represent a set of relationships between a set of biological molecules, wherein each graph can comprise vertices representing the biological molecules and edges representing the relationships between the biological molecules, where the computer program product comprises a computer data medium on which is carried a means for performing one or more operations on the one or more graphs to produce one or more product graphs.

Another embodiment of the disclosed composition is a computer program product for performing an operation upon a graph, where the graph can represent relationships between biological molecules and can have vertices representing the molecules and edges representing the relationships, where the computer program product comprises a computer data medium on which is carried a means for identifying a subset of zero or more of the edges, a means for identifying a subset of zero or more of the vertices, and a means for performing a unary operation upon the identified subset of edges and vertices to produce a product graph.

Another embodiment of the disclosed composition is a computer program product for representing relationships between biological molecules using a graph having vertices and edges, where the computer program product comprises a computer data medium on which is carried a means for representing a set of biological molecules, wherein each molecule can be represented by a vertex of the graph, and a means for representing a set of relationships between the biological molecules, wherein each relationship can be represented by an edge of the graph, wherein the edge connects two vertices.

Another embodiment of the disclosed method is a computer-implemented method for representing relationships between biological molecules using a graph having vertices and edges, where the method comprises representing a set of biological molecules, wherein each molecule can be represented by a vertex of the graph, and representing a set of relationships between the biological molecules, wherein each relationship can be represented by an edge of the graph, wherein the edge connects two vertices.

Another embodiment of the disclosed composition is a representation of relationships between biological molecules comprising one or more graphs each having vertices and edges, each graph comprising a set of biological molecules, wherein each molecule can be represented by a vertex of the graph, and a set of relationships between the biological molecules, wherein each relationship can be represented by an edge of the graph, wherein the edge connects two vertices, wherein the graph can be produced by performing one or more operations on one or more input graphs to produce the one or more graphs.

Another embodiment of the disclosed composition is a data structure comprising a representation of relationships between biological molecules, where the representation can comprise a graph having vertices and edges, where the graph comprises a set of biological molecules, wherein each molecule can be represented by a vertex of the graph, and a set of relationships between the biological molecules, wherein each relationship can be represented by an edge of the graph, wherein the edge connects two vertices. A data structure is any form of data, information, and/or objects collected, organized, stored, and/or embodied in a composition or medium. A molecular relational graph stored in electronic form, such as in RAM or on a storage disk, is a type of data structure.

Another embodiment of the disclosed method is a computer-implemented method for graphically representing relationships between biological molecules using a graph having vertices and edges, where the method comprises displaying a representation of a set of biological molecules, where each molecule can be graphically represented by a vertex of the graph; and displaying a representation of a set of relationships between the molecules, where each relationship can be graphically represented by an edge of the graph, where each edge can have an associated description, wherein the edge connects two vertices. As used herein, a graphical representation is a visual representation of a graph.

Another embodiment of the disclosed method is a computer-implemented method for performing an operation upon a graph, where the graph can represent relationships between biological molecules and can have vertices representing the molecules and edges representing the relationships, where the method comprises displaying the graph; identifying a subset of zero or more of the edges; identifying a subset of zero or more of the vertices; performing a unary operation upon the identified subset of edges and vertices; and displaying a product graph resulting from the unary operation.

Another embodiment of the disclosed method is a computer-implemented method for performing an operation upon a set of n graphs, where each graph can represent relationships between biological molecules and can have vertices representing the molecules and edges representing the relationships, where the method comprises performing an n-nary operation upon the n graphs; and displaying a product graph resulting from the n-nary operation.

Another embodiment of the disclosed composition is a computer program product for graphically representing relationships between biological molecules using a graph having vertices and edges, where the computer program product comprises a computer data medium on which is carried a means for displaying a representation of a set of biological molecules, where each molecule can be graphically represented by a vertex of the graph; and a means for displaying a representation of a set of relationships between the molecules, where each relationship can be graphically represented by an edge of the graph, each edge having an associated description.

In these or other embodiments disclosed herein, the method or composition can have any or a combination of the following features. For example, the operations can comprise finding a common subset of vertices and edges in a plurality of graphs; merging a plurality of graphs having one or more common vertices or edges; deleting vertices and edges present in a first graph that are not present in a second graph; combining the edges and vertices of a plurality of graphs; finding a common subset of vertices and edges present in a predetermined percent of a plurality of graphs; finding a common subset of vertices and edges in a plurality of graphs, and deleting the common subset of vertices and edges from each of the graphs to produce a plurality of graphs each with a unique set of vertices and edges; deleting all edges beyond a selected range of edge weights; dividing one graph into two graphs; using an AND operation to find the common subsets of vertices and edges of n graphs; or any combination of these and/or other operations. Any of the operations can be a recursive operation.

The set of biological molecules can comprise more than one type of biological molecule or can be all of the same type of biological molecule. The biological molecules can be, for example, selected from the group consisting of genes, open reading frames, expressed sequence tags, single nucleotide polymorphisms, sequence tag sites, nucleic acids, DNA, RNA, mRNA, cDNA, proteins, peptides, enzymes, metabolites, carbohydrates, exons, introns, cleavage fragments, restriction fragments, amino acid modifications, protein domains, DNA or RNA secondary or tertiary structures, nucleic acid motifs, protein motifs, and metal ions.

The set of relationships can comprise more than one type of relationship or can be all of the same type of relationship. The relationships can be, for example, selected from the group consisting of physical distances between genes, open reading frames, single nucleotide polymorphisms, expressed sequence tags, sequence tag sites, or a combination thereof; genetic distances between genes, open reading frames, single nucleotide polymorphisms, expressed sequence tags, sequence tag sites, or a combination thereof; protein-protein interactions; protein-nucleic acid interactions; gene expression regulation; protein expression regulation; cellular signal transduction pathways; sequence similarity between genes or proteins; structural similarity between proteins; radiation hybrid mapping distances between genes, open reading frames, single nucleotide polymorphisms, expressed sequence tags, sequence tag sites, or a combination thereof; and metabolic pathways.

The edges can have a variety of values and features. For example, at least one edge can comprise a direction; at least one edge can comprise a boolean value indicating the presence or absence of an association between the biological molecules represented by the vertices connected by the edge (where, in some embodiments, the association can be co-expression, co-regulation, or presence or use in the same pathway); at least two of the vertices can represent different types of biological molecules; at least two edges can represent different types of relationships between the biological molecules represented by the vertices connected by the edges; at least one edge can represent a plurality of different types of relationships between the biological molecules represented by the vertices connected by the edge; at least one vertex can represent a plurality of different biological molecules; at least one edge can comprise an edge weight; a subset of edges can be edges beyond a selected range of edge weights; or any combination of these and/or other features.

Where an edge comprises an edge weight, the edge weight can represent a value characterizing the relationship represented by the edge (where, in some embodiments, the value can be a numerical value; at least one edge can comprise an edge weight table comprising the edge weight (where, in some embodiments, the edge weight table further can comprise one or more additional edge weights); at least one edge weight can comprise an indication of a state; at least one edge weight can comprise a spatial distance (where, in some embodiments, the spatial distance can represent a physical distance between the biological molecules represented by the vertices connected by the edge); at least one edge weight can comprise a kinetic measurement; at least one edge weight can comprise a distance metric representing a logical relationship between the biological molecules represented by the vertices connected by the edge; at least one edge weight can comprise a statistical metric representing a logical relationship between the biological molecules represented by the vertices connected by the edge; at least one edge weight can comprise a value of fuzzy set membership representing a logical relationship between the biological molecules represented by the vertices connected by the edge; at least one edge weight can comprise a conditional probability (where, in some embodiments, the conditional probability can be the probability of a causal relationship between the biological molecules represented by the vertices connected by the edge); or any combination of these and/or other features.

The disclosed method and compositions can also comprise hyper-edges and/or hyper-vertices. For example, at least one of the graphs can comprise at least one hyper-edge (where, in some embodiments, at least one of the operations can convert at least one hyper-edge to a non-hyper-edge); at least one of the graphs can comprise at least one hyper-vertex (where, in some embodiments, at least one of the operations can convert at least one hyper-vertex to a non-hyper-vertex); at least one of the graphs can comprise at least one hyper-edge and at least one hyper-vertex (where, in some embodiments, at least one of the operations can convert at least one hyper-edge to a non-hyper-edge, at least one of the operations can convert at least one hyper-vertex to a non-hyper-vertex, and/or at least one of the operations can convert at least one hyper-edge to a non-hyper-edge and at least one hyper-vertex to a non-hyper-vertex); at least one of the operations can convert at least one edge to a hyper-edge (where, in some embodiments, the hyper-edge can be formed by combining two or more edges); at least one of the operations can convert at least one vertex to a hyper-vertex (where, in some embodiments, the hyper-vertex can be formed by combining two or more vertices; at least one of the operations can convert at least one edge to a hyper-edge and at least one vertex to a hyper-vertex (where, in some embodiments, the hyper-edge can be formed by combining two or more edges and the hyper-vertex is formed by combining two or more vertices); or any combination of these and/or other features.

The product graph produced or present in any embodiment of the disclosed method or composition can be a graph that is modified relative to the graph on which the operation is performed.

As indicated above, the disclosed methods can be performed using a suitable computer or other electronic system. In the illustrated embodiment of the invention, the methods can be performed using a suitably programmed general-purpose computer system such as that illustrated in FIG. 14. Persons skilled in the art to which the invention pertains will readily be capable of programming the computer system or otherwise providing it with suitable software to implement the above-described methods.

Although the software can be structured in any suitable manner and written in any suitable programming languages, it can be conceptually considered to include a GGO subsystem 102, and a data mining service broker 104. This software executes in the memory 106 of the computer in the manner in which application software conventionally executes in such computers. Although GGO subsystem 102 and data mining service broker 104 are conceptually illustrated as residing in memory 106 for purposes of clarity, persons of skill in the art will recognize that in actual operation they may not reside in memory 106 simultaneously or in their entireties. Such persons will further understand that many other software elements that typically execute in such a computer system, such as operating system software, network communication software, software utilities, and other application programs are not illustrated for purposes of clarity.

In addition to memory 106, the computer system can include other suitable hardware that is typically included in a general purpose computer, such as a processor 108, a network interface 110, a fixed-medium disk drive 112 such as a hard disk drive, a removable-medium disk drive 114 such as a floppy disk or optical disk drive, and input/output interface logic 116. The software elements described that embody a system of the present invention can be provided via a program product, such as a floppy disk 118 on which such elements are recorded. Alternatively, the can be provided via a network 120 from a remote site. The software elements can be transferred to disk drive 112 for long-term storage, from where they are used during operation of the system by loading them into memory 106 as needed, under the control of processor 108, in the manner well-understood in the art.

The user can interact with the computer system using a mouse 122, keyboard 124 and video monitor or other display 126 in the conventional manner. Thus, where it is described above that the user makes a selection or otherwise provides input in response to a displayed menu or other output, such steps can be implemented by using mouse 122 and keyboard 124 to provide input in response to information output on display 126. Note that descriptions above of outputting graphs for the user refer in the illustrated embodiment of the invention to displaying them on display 126. Although not illustrated for purposes of clarity, the graphs can alternatively be output to a printer (not shown) or any other suitable output device or sent to a remote system via network 120. Likewise, graphs can be received from such a remote system via network 120 or input via any other suitable input device, such as disk 118. Furthermore, as described below, users of remote systems can use the illustrated system for data mining purposes.

As illustrated in further detail in FIG. 6, GGO subsystem 102 can include a graph computation manager 130, a graph visualization engine 132, a graph computation engine 134 and a graph database 136. Graph computation manager 130 can interface not only with graph database 136 but also with other inside databases 140 and outside databases 142. Graph computation manager 130 also interfaces with data mining service broker 104. The other inside databases can be databases containing representations of genes, open reading frames, expressed sequence tags, single nucleotide polymorphisms, sequence tag sites, nucleic acids, DNA, RNA, mRNA, cDNA, proteins, peptides, enzymes, metabolites, carbohydrates, exons, introns, cleavage fragments, restriction fragments, amino acid modifications, protein domains, DNA or RNA secondary or tertiary structures, nucleic acid motifs, protein motifs, and metal ions. The other inside databases can also contain information about the sample collection and experimental processing of the biological materials as captured by a Laboratory Information Management System, LIMS.

Graph computation manager 130 is a middleware component or element that performs data mining, visualizes results of data mining, queries previous data mining results, and visualizes result data. Graph computation engine 134 is a toolkit/library that provides ways to construct graphs and perform graph computations. Graph visualization engine 132 creates graphics objects from graph data objects.

Data mining service broker 104 is a middleware component that communicates with a data mining service client 100, decomposes data mining request objects, dispatches requests to appropriate subsystems, and receives computational or database querying result objects and sends them to data mining service client.

As illustrated in FIG. 7, data mining service client 100 can include a graphical user interface (GUI) 150, a request constructor 152, a result unbundler 154, and a communications interface 156.

As illustrated in FIG. 8, data mining service broker 104 can include a client manager 160, a client queue 162, a request dispatcher 164, a result dispatcher 166, and communications interfaces 167, 168, and 169.

As illustrated in FIG. 9, graph computation manager 130 can include a job manager 170, a job queue 172, a graph computational organizer 174, an outside database query engine 176, an other inside database query engine 178, a graph database engine 180, a graph visualization unit, and communications interfaces 184, 185, 186, 187, 188, and 189.

As illustrated in FIG. 10, graph computation engine 134 can include graph computation engine 190, which can include graph computation executor 192 and graph computation library 194, and communications interface 196.

As illustrated in FIG. 11, graph visualization engine 132 can include a graph visualization constructor 200 and a communications interface 202. Tom Sawyer GLT 3.1, referred to in FIGS. 6 and 11, is only an example of graphical representation software that can be used in the graph visualization engine.

As illustrated in FIG. 12, graph computation library 194 can include gene graph operator 196, which can include strict graph 198.

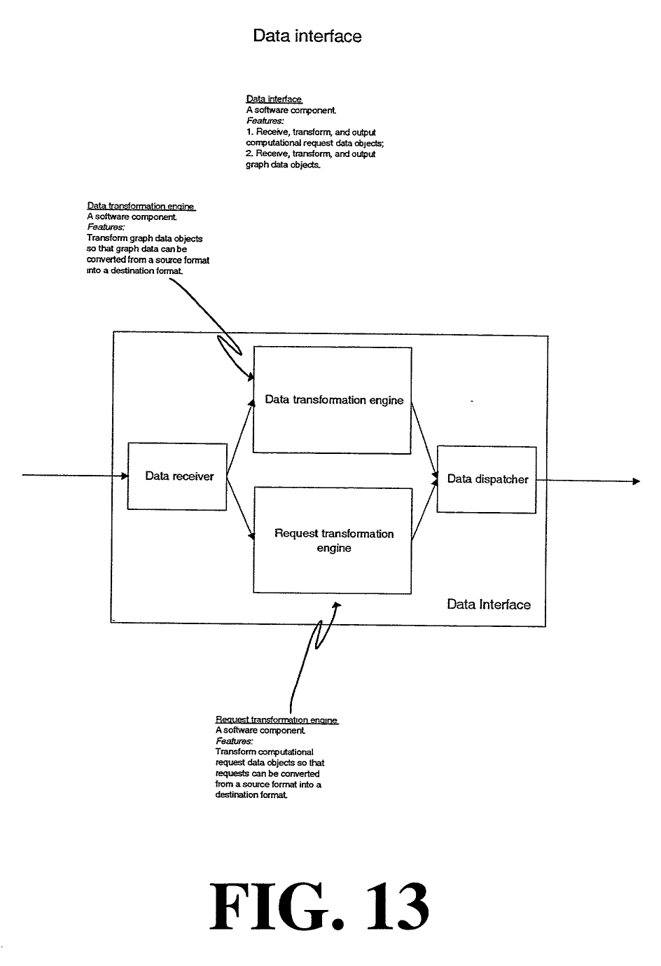

As illustrated in FIG. 13, data interface 210 can include a data receiver 212, a data transformation engine 214, a request transformation engine 216, and a data dispatcher 218.

### EXAMPLES

An example of the disclosed method involving a molecular relational graph of genomics data has been implemented using the Java programming language. Software has been developed to convert genomics information to graph structure. Using the programs, data from microarray gene expression assays, protein-protein interaction assays, and Gene Ontology functional annotation (Gene Ontology consortium, 1998) have been encoded into graph structures. A set of graph visualization tools is incorporated into the programs.

Data was imported from the analysis of the yeast (Saccharomyces cerevisiae) genome, and these data were encoded into molecular relational graphs. As shown in FIG. 2, the 1,004 yeast genes and 957 protein-protein interactions documented by Uetz et al. (2000) have been graphed. The resulting graph shows structural complexities, such as the subset of strongly connected components seen in the middle of FIG. 2. Similarly, for another data set, data derived from the Gene Ontology (GO) annotation for functional relationships of a selected set of yeast genes was encoded. The graph shown in FIG. 3 was generated by connecting genes that share the same unique GO functional identifier. This graph clearly shows known functional relationships of the yeast genes. More importantly, from inspection of the molecular relational graph, higher-order functional gene relationships not previously characterized can be deduced.

Quantitative relational data such as correlation coefficients also can be represented in graph form. Microarray hybridization data for gene expression during the yeast cell cycle (Spellman et al., 1998) was analyzed. The correlation coefficients for the expression profile of a selected set of gene pairs were computed and used as a metric to define the edge weight for the edges connecting each pair of genes. The resulting molecular relational graphing (not shown) is a completely connected graph in which each vertex is connected to every other vertex. The edges of this graph are weighted by the correlation coefficients. However, a “threshold” operation can be performed on the edges of the graph to produce a less densely connected graph depicting only the stronger relationships. A threshold of 0.6 was used, where a value of 0 corresponds to no correlation, and a value of 1 to complete correlation. In this threshold operation, edges were deleted if their weights are less than or equal to 0.6. The resulting graph is shown in FIG. 4. This operation reveals the expression relationships between genes, graded by a degree of confidence. The degree of confidence is determined by the threshold parameter.

A strength of the disclosed molecular relational graphing model comes from the ability to manipulate and combine graphs. In order to demonstrate this capability, a small number of graph operators for the molecular relational graphing data model were defined, including add vertex, delete vertex, add edge, delete edge, threshold edges, convert graph, subset, graph AND, and graph OR. These operators were implemented in the example software.

The molecular relational graph of the complete set of GO functional relationships, and the molecular relational graph of expression data shown in FIG. 4 were used to illustrate graph manipulations. The graph of GO functional relationships is an unweighted graph, while the graph in FIG. 4 is a weighted graph, in which the edge weights are the correlation coefficients. The unary operator “convert” transforms a graph from one type to another, so that graphs from different sources can be compared. The “convert” operator was used to transform the weighted graph shown in FIG. 4 to an unweighted graph (not shown).

The binary operator “AND” synthesizes information from two or more graphs by finding the subset of common edges and vertices. The “AND” operator was applied to the complete set of GO functional relationships (not shown) and the molecular relational graph of a subset of data from the expression study of Spellman et al. (1998), (shown in FIG. 4). FIG. 5A depicts this synthesis of information. Because only a subset of the 6,000+ yeast genes was used to generate FIG. 4, the results shown in FIG. 5A are merely illustrative, and do not represent an exhaustive survey. FIG. 5A shows two connected component structures representing two distinct sets of genes. These sets represent those genes whose GO functional relationships are concordant with their expression pattern relationships.

Additional threshold operations were used on the graph in FIG. 4 to determine whether stronger correlations in gene expression are related to functional relationships. That is, it was asked whether the structure shown in FIG. 5A can be recovered from the graph shown FIG. 4 alone by subsetting only the strongest pattern relationships. Both of the connected components seen in FIG. 5A appear in expression molecular relational graphs thresholded at 0.9 (FIG. 5B), 0.8 (FIG. 5C), and 0.7 (FIG. 5D). Higher-stringency thresholding produces fewer gene-relationship structures in the expression data, but more of the structures produced are supported by the GO data. This suggests a quantitative relationship between concordant expression of genes and their functional interaction. In addition, FIG. 5 shows that the expression data also imply some gene relationships (marked by ∇ in FIGS. 5B, 5C, and 5D) which are not apparent in the GO molecular relational graph (FIG. 3). Careful examination shows that a higher-order relationship documented in the GO tree can account for these expression relationships (FIG. 5E). This exercise demonstrates how a novel inference can be made through the power of integrative analysis using the disclosed molecular relational graphing data model. Operations used to generate FIG. 5 are summarized in Table 4.

TABLE 5
Operation used to generate the molecular relational
graphs shown in FIG 5.

| | | | Resulting |
|---|---|---|---|
| Graph A | Graph B | Operator | Graph |
| GO graph | Expression graph | AND | |
| | Expression graph | Threshold at 0.9 | |
| | Expression graph | Threshold at 0.8 | |
| | Expression graph | Threshold at 07 | FIG. 5D |

In summary, the disclosed molecular relational graphing provides a powerful tool for the analysis of large genomic data sets and for the discovery of novel gene relationships. In addition, it provides an elegant method for the corroboration of relational data by drawing consensus from disparate sources of information. Further enrichment of the algorithmic operations on the molecular relational graph by adding new theoretical and heuristic operators can greatly expand the potential of this analytical technique, and transform it into a significant discovery tool for genome-scale data analysis.

### References

Bairoch, (2000) The Enzyme Database in 2000. Nucleic Acids Research, 28:304-305

Bergeron et al., (1997) Combinatorial species and tree-like structures. Cambridge University Press, New York.

Boguski et al., (1999) Biosequence Exegesis. Science, 286(5439):453-455.

Brown and Botstein, (1999) Exploring the new world of the genome with DNA microarrays. Nature Genetics, 21 (1 Suppl):33-7.

Chan et al., (1999) Microfabricated polymer devices for automated sample delivery of peptides for analysis by electrospray ionization tandem mass spectrometry. Analytical Chemistry, 71(20):4437-44.

Cherry et al., (1997) Genetic and physical maps of Saccharomyces cerevisiae, Nature, 387(6632 Suppl.):67-73.

Cherry et al., “Saccharomyces Genome Database”, http://genome-www.stanford.edu/Saccharomyces/.

Eisen et al., (1998) Cluster analysis and display of genome-wide expression patterns. Proceedings of the National Academy of Sciences of the United States of America 95(25):14863-8.

Forst and Schulten (1999) Evolutoin of Metabolisms: A new method for the comparison of metabolic pathways using genomics information. Journal of Computational Biology, 6:343-360.

The Gene Ontology Consortium, (2000) Gene Ontology: tool for the unification of biology. Nature Genetics, 25: 25-29.

Graves et al., (1995) A Graph-Theoretic Data Model for Genomic Mapping Databases. Proceedings of the 28^{th} Annual Hawaii International Conference on System Sciences, 5:32-41.

Kanehisa and Susumu, (2000) KEGG: Kyoto Encyclopedia of Genes and Genomes. Nucleic Acids Research, 28(1):27-30.

Koch and Lengauer, (1997) Detection of distant structural similarities in a set of proteins using a fast graph-based method. ISMB, 5:167-78.

Minieka, (1978) Optimization algorithms for networks and graphs. Marcel Dekker, Inc, New York.

Ore, (1962) Theory of graphs. American Mathematical Society, Providence, RI.

Patton, (2000) Making blind robots see: the synergy between fluorescent dyes and imaging devices in automated proteomics. Biotechniques, 28(5):944-8, 950-7

Robinson and Foulds, (1979) Comparison of weighted labelled trees, Lecture Notes in Mathematics, Vol. 748, pp. 119-126. Springer-Verlag, Berlin.

Robinson, (1971) Comparison of labeled trees with valency three, Journal of Combinatorial Theory, 11:105-119

Rohlf, (1982) Consensus indices for comparing classifications. Math. Biosci., 59:313-144.

Samudrala and Moult, (1998) A Graph-theoretic Algorithm for Comparative Modeling of Protein Structure. Journal of Molecular Biology, 279:287-302.

Steel and Penny, (1993) Distributions of tree comparison metrics. Systematic Biology, 42:126-141.

Spellman et al., (1998) Comprehensive identification of cell cycle-regulated genes of the yeast Saccharomyces cerevisiae by microarray hybridization. Mol Biol Cell, 9(12):3273-97.

The Gene Ontology Consortium (2000) Gene Ontolog: tool for the unification of biology. Nature Genetics, 25: 25-29.

Toba et al., (1999) The Gene Search System: A method for efficient detection and rapid molecular identification of genes in Drosophila melanogaster. Genetics, 151:725-737.

Uetz et al., (2000) A comprehensive analysis of protein-protein interactions in Saccharomyces cerevisiae. Nature, 403(6770):623-7.

It is understood that the disclosed invention is not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a ”, “an”, and “the” include plural reference unless the context clearly dictates otherwise. Thus, for example, reference to “a host cell” includes a plurality of such host cells, reference to “the antibody” is a reference to one or more antibodies and equivalents thereof known to those skilled in the art, and so forth.

Unless defined otherwise, all technical and scientific terms used herein have the same meanings as commonly understood by one of skill in the art to which the disclosed invention belongs. Although any methods and materials similar or equivalent to those described herein can be used in the practice or testing of the present invention, the preferred methods, devices, and materials are as described. Publications cited herein and the material for which they are cited are specifically incorporated by reference. Nothing herein is to be construed as an admission that the invention is not entitled to antedate such disclosure by virtue of prior invention.

Those skilled in the art will recognize, or be able to ascertain using no more than routine experimentation, many equivalents to the specific embodiments of the invention described herein. Such equivalents are intended to be encompassed by the following claims.

We claim:

1. A computer-implemented method for performing an operation upon one or more graphs, wherein each graph represents a set of relationships between a set of biological molecules, wherein each graph comprises vertices representing the biological molecules and edges representing the relationships between the biological molecules, the method comprising
performing one or more operations on the one or more graphs to produce one or more product graphs.

2. The method of claim 1 wherein the operations comprise
finding a common subset of vertices and edges in a plurality of graphs.

3. The method of claim 1 wherein the operations comprise
merging a plurality of graphs having one or more common vertices or edges.

4. The method of claim 1 wherein the operations comprise
deleting vertices and edges present in a first graph that are not present in a second graph.

5. The method of claim 1 wherein the operations comprise
combining the edges and vertices of a plurality of graphs.

6. The method of claim 1 wherein the operations comprise
finding a common subset of vertices and edges present in a predetermined percent of a plurality of graphs.

7. The method of claim 1 wherein the operations comprise
finding a common subset of vertices and edges in a plurality of graphs,
deleting the common subset of vertices and edges from each of the graphs to produce a plurality of graphs each with a unique set of vertices and edges.

8. The method of claim 1 wherein the operation is a recursive operation.

9. The method of claim 1 wherein the set of biological molecules comprises more than one type of biological molecule.

10. The method of claim 1 wherein the set of relationships comprises more than one type of relationship.

11. The method of claim 1 wherein at least one edge comprises an edge weight.

12. The method of claim 11 wherein the edge weight represents a value characterizing the relationship represented by the edge.

13. The method of claim 12 wherein the value is a numerical value.

14. The method of claim 11 wherein at least one edge comprises an edge weight table comprising the edge weight.

15. The method of claim 14 wherein the edge weight table further comprises one or more additional edge weights.

16. The method of claim 11 wherein at least one edge weight comprises an indication of a state.

17. The method of claim 11 wherein at least one edge weight comprises a spatial distance.

18. The method of claim 17 wherein the spatial distance represents a physical distance between the biological molecules represented by the vertices connected by the edge.

19. The method of claim 11 wherein at least one edge weight comprises a kinetic measurement.

20. The method of claim 11 wherein at least one edge weight comprises a distance metric representing a logical relationship between the biological molecules represented by the vertices connected by the edge.

21. The method of claim 11 wherein at least one edge weight comprises a statistical metric representing a logical relationship between the biological molecules represented by the vertices connected by the edge.

22. The method of claim 11 wherein at least one edge weight comprises a value of fuzzy set membership representing a logical relationship between the biological molecules represented by the vertices connected by the edge.

23. The method of claim 11 wherein at least one edge weight comprises a conditional probability.

24. The method of claim 23 wherein the conditional probability is the probability of a causal relationship between the biological molecules represented by the vertices connected by the edge.

25. The method of claim 1 wherein at least one edge comprises a direction.

26. The method of claim 1 wherein at least one edge comprises a boolean value indicating the presence or absence of an association between the biological molecules represented by the vertices connected by the edge.

27. The method of claim 26 wherein the association is co-expression, co-regulation, or presence or use in the same pathway.

28. The method of claim 1 wherein the biological molecules are selected from the group consisting of genes, open reading frames, expressed sequence tags, single nucleotide polymorphisms, sequence tag sites, nucleic acids, DNA, RNA, mRNA, cDNA, proteins, peptides, enzymes, metabolites, carbohydrates, exons, introns, cleavage fragments, restriction fragments, amino acid modifications, protein domains, DNA or RNA secondary or tertiary structures, nucleic acid motifs, protein motifs, and metal ions.

29. The method of claim 1 wherein at least two of the vertices represent different types of biological molecules.

30. The method of claim 1 wherein at least two edges represent different types of relationships between the biological molecules represented by the vertices connected by the edges.

31. The method of claim 1 wherein at least one edge represents a plurality of different types of relationships between the biological molecules represented by the vertices connected by the edge.

32. The method of claim 1 wherein the relationships are selected from the group consisting of physical distances between genes, open reading frames, single nucleotide polymorphisms, expressed sequence tags, sequence tag sites, or a combination thereof; genetic distances between genes, open reading frames, single nucleotide polymorphisms, expressed sequence tags, sequence tag sites, or a combination thereof; protein-protein interactions; protein-nucleic acid interactions; gene expression regulation; protein expression regulation; cellular signal transduction pathways; sequence similarity between genes or proteins; structural similarity between proteins; radiation hybrid mapping distances between genes, open reading frames, single nucleotide polymorphisms, expressed sequence tags, sequence tag sites, or a combination thereof; and metabolic pathways.

33. The method of claim 1 wherein at least one of the graphs comprises at least one hyper-edge.

34. The method of claim 33 wherein at least one of the operations converts at least one hyper-edge to a non-hyper-edge.

35. The method of claim 1 wherein at least one of the graphs comprises at least one hyper-vertex.

36. The method of claim 35 wherein at least one of the operations converts at least one hyper-vertex to a non-hyper-vertex.

37. The method of claim 1 wherein at least one of the graphs comprises at least one hyper-edge and at least one hyper-vertex.

38. The method of claim 37 wherein at least one of the operations converts at least one hyper-edge to a non-hyper-edge.

39. The method of claim 37 wherein at least one of the operations converts at least one hyper-vertex to a non-hyper-vertex.

40. The method of claim 37 wherein at least one of the operations converts at least one hyper-edge to a non-hyper-edge and at least one hyper-vertex to a non-hyper-vertex.

41. The method of claim 1 wherein at least one of the operations converts at least one edge to a hyper-edge.

42. The method of claim 41 wherein the hyper-edge is formed by combining two or more edges.

43. The method of claim 1 wherein at least one of the operations converts at least one vertex to a hyper-vertex.

44. The method of claim 43 wherein the hyper-vertex is formed by combining two or more vertices.

45. The method of claim 1 wherein at least one of the operations converts at least one edge to a hyper-edge and at least one vertex to a hyper-vertex.

46. The method of claim 45 wherein the hyper-edge is formed by combining two or more edges and the hyper-vertex is formed by combining two or more vertices.

47. The method of claim 1 wherein the product graph is modified relative to the graph on which the operation is performed.

48. The method of claim 1 wherein the operations comprise
delete all edges beyond a selected range of edge weights.

49. The method of claim 1 wherein the operations comprise
dividing one graph into two graphs.

50. A computer-implemented method for performing an operation upon a graph, the graph representing relationships between biological molecules and having vertices representing the molecules and edges representing the relationships, the method comprising
identifying a subset of zero or more of the edges,
identifying a subset of zero or more of the vertices, and
performing a unary operation upon the identified subset of edges and vertices to produce a product graph.

51. The method of claim 50 wherein the subset of edges identified are all edges beyond a selected range of edge weights.

52. A computer-implemented method for representing relationships between biological molecules using one or more graphs each having vertices and edges, the method comprising
representing a set of biological molecules, wherein each molecule is represented by a vertex of the graph, and
representing a set of relationships between the biological molecules, wherein each relationship is represented by an edge of the graph, wherein the edge connects two vertices,
wherein the graph is produced by performing one or more operations on one or more input graphs to produce the one or more graphs.

53. A computer program product for performing an operation upon one or more graphs, wherein each graph represents a set of relationships between a set of biological molecules, wherein each graph comprises vertices representing the biological molecules and edges representing the relationships between the biological molecules, the computer program product comprising a computer data medium on which is carried
a means for performing one or more operations on the one or more graphs to produce one or more product graphs.

54. A computer program product for performing an operation upon a graph, the graph representing relationships between biological molecules and having vertices representing the molecules and edges representing the relationships, the computer program product comprising a computer data medium on which is carried
a means for identifying a subset of zero or more of the edges,
a means for identifying a subset of zero or more of the vertices, and
a means for performing a unary operation upon the identified subset of edges and vertices to produce a product graph.

55. A computer program product for representing relationships between biological molecules using a graph having vertices and edges, the computer program product comprising a computer data medium on which is carried
a means for representing a set of biological molecules, wherein each molecule is represented by a vertex of the graph, and
a means for representing a set of relationships between the biological molecules, wherein each relationship is represented by an edge of the graph, wherein the edge connects two vertices.

56. A computer-implemented method for representing relationships between biological molecules using a graph having vertices and edges, the method comprising
representing a set of biological molecules, wherein each molecule is represented by a vertex of the graph, and
representing a set of relationships between the biological molecules, wherein each relationship is represented by an edge of the graph, wherein the edge connects two vertices.

57. A representation of relationships between biological molecules comprising one or more graphs each having vertices and edges, each graph comprising
a set of biological molecules, wherein each molecule is represented by a vertex of the graph, and
a set of relationships between the biological molecules, wherein each relationship is represented by an edge of the graph, wherein the edge connects two vertices,
wherein the graph is produced by performing one or more operations on one or more input graphs to produce the one or more graphs.

58. The representation of claim 57 wherein the set of biological molecules comprises more than one type of biological molecule.

59. The representation of claim 57 wherein the set of relationships comprises more than one type of relationship.

60. A data structure comprising a representation of relationships between biological molecules, the representation comprising a graph having vertices and edges, the graph comprising
a set of biological molecules, wherein each molecule is represented by a vertex of the graph, and
a set of relationships between the biological molecules, wherein each relationship is represented by an edge of the graph, wherein the edge connects two vertices.

61. A computer-implemented method for performing an operation upon one or more graphs, wherein each graph represents a set of relationships between a set of biological molecules, wherein each graph comprises vertices representing the biological molecules and edges representing the relationships between the biological molecules, wherein the biological molecules, the relationships between the biological molecules, or both, are derived from different sources, the method comprising
performing one or more operations on the one or more graphs to produce one or more product graphs.

62. A computer-implemented method for performing an operation upon one or more graphs, wherein each graph represents a set of relationships between a set of biological molecules, wherein each graph comprises vertices representing the biological molecules and edges representing the relationships between the biological molecules,
wherein at least two of the vertices represent different types of biological molecules, at least two edges represent different types of relationships between the biological molecules represented by the vertices connected by the edges, at least one edge represents a plurality of different types of relationships between the biological molecules represented by the vertices connected by the edge, at least one vertex represents a plurality of different types of biological molecules, or a combination thereof,
the method comprising
performing one or more operations on the one or more graphs to produce one or more product graphs.

63. A computer-implemented method for performing an operation upon one or more graphs, wherein each graph represents a set of relationships between a set of biological molecules, wherein each graph comprises vertices representing the biological molecules and edges representing the relationships between the biological molecules, wherein the biological molecules, the relationships between the biological molecules, or both, are derived from heterogeneous molecular biological data, the method comprising
performing one or more operations on the one or more graphs to produce one or more product graphs.
